(19) United States
(12) Patent Application Publication
(10) Pub. No.: US 2026/0070985 A1
(43) Pub. Date: Mar. 12, 2026

(54) ENHANCED IMMUNE CHECKPOINT BLOCKADES FORMULATION FOR IMAGE GUIDED LOCAL IMMUNOTHERAPY

(71) Applicant: Northwestern University, Evanston, IL (US)

(72) Inventors: Dong Hyun Kim, Evanston, IL (US); Bongseo Choi, Evanston, IL (US)

(21) Appl. No.: 18/707,730

(22) PCT Filed: Nov. 7, 2022
(86) PCT No.: PCT/US2022/079377

§ 371 Date: May 6, 2024

Related U.S. Application Data
(60) Provisional application No. 63/276,213, filed on Nov. 5, 2021.

(51) Int. Cl. C07K 16/28; A61K 38/00; A61K 47/69; A61P 35/00

(52) U.S. Cl. CPC C07K 16/2827; A61K 38/00; A61K 47/6923; A61K 47/6929; A61P 35/00; C07K 2317/52; C07K 2317/73; C07K 2317/92

(57) ABSTRACT

Disclosed are nanoparticles comprising directionally attached antibodies and compositions for use in locoregional delivery, including intra-tumoral and transarterial chemoembolization (TACE), and methods of making the nanoparticles. Also disclosed are methods for treating a subject in need thereof the compositions described.

### CROSS-REFERENCE TO RELATED APPLICATIONS

This application claims priority to, and the benefit of, U.S. Application No. 63/276,213 filed Nov. 5, 2021, the content of which is incorporated herein by reference in its entirety.

### STATEMENT REGARDING FEDERALLY SPONSORED RESEARCH

This invention was made with government support under R01CA218659, R01EB026207, and R01CA218659 awarded by the National Institutes of Health. The Government has certain rights in the invention.

### SEQUENCE LISTING

A Sequence Listing accompanies this application and is submitted as an ASCII text file of the sequence listing named “702581_02035_ST26.txt” which is 6,099 bytes in size and was created on Nov. 4, 2021. The sequence listing is electronically submitted via EFS-Web with the application and is incorporated herein by reference in its entirety.

### BACKGROUND

Current systemic administration of immune checkpoint inhibitor (ICI) cancer immunotherapy has not shown remarkable therapeutic outcomes in the clinic yet. Balancing the ICI immune activation and immune-related adverse effects (irAEs) has been challenging. Local immunotherapies including peritumoral and intratumoral immune modulations have shown potential for enhanced local anti-cancer immunity with minimized irAEs. In the case of immune-suppressive hepatocellular carcinoma (HCC), local ICI immunotherapy might be essential to overcome ICI immunotherapy resistance, immune tolerance, and irAEs.

### SUMMARY OF THE INVENTION

In a first aspect, the present invention provides a nanoparticle comprising an antibody directionally attached by the constant region (Fc) of the antibody to an iron nanoparticle with a directional linker, wherein the antigen binding sites of the antibody are facing outwardly from the nanoparticle. In some embodiments, the nanoparticle comprises ferumoxytol. In some embodiments, the antibody is an immune checkpoint inhibitor (ICI) antibody. In some embodiments, the nanoparticle surface is functionalized with free exposed carboxyl residues chemically linked to glutathione. In some embodiments, the linker comprises glutathione S transferase (GST) linked to the Z domain of staphylococcal protein A. In some embodiments, the ICI is an anti-PD-L1 or anti-PD-1 antibody.

In some embodiments, the nanoparticle has a greater binding affinity for the antigen, an increased resistance to fluidic shear force, induces increased CD3+ cell accumulation in a tumor microenvironment (TME), reduces myeloid derived suppressor cell (MDSC) accumulation in a TME, and/or increases dendritic cell (DC) maturation compared to the antibody alone or a second iron nanoparticle having the antibody chemically linked directly to the second nanoparticle.

In another aspect, the disclosure provides a pharmaceutical composition comprising any of the disclosed nanoparticles and a pharmaceutically acceptable delivery vehicle.

In another aspect, the disclosure provides a method of treating a subject in need of treatment for cancer, the method comprising administering an effective amount of the disclosed pharmaceutical composition. In some embodiments, the cancer is hepatocellular carcinoma (HCC). In some embodiments, the administering is locoregionally. In some embodiments, the administering is transarterially, intratumorally, or intrahepactically.

In another aspect, the disclosure provides a method comprising conjugating glutathione to an iron nanoparticle; co-incubating the nanoparticle with a glutathione S-transferase functionalized with a Z domain of staphylococcal protein A; and co-incubating the nanoparticle with an antibody. In some embodiments, the iron nanoparticle comprises ferumoxytol. In some embodiments, prior to the conjugating step, the nanoparticle is functionalized with free exposed carboxyl residues. In some embodiments, the antibody is an ICI antibody. In some embodiments, the ICI antibody is an anti-PD-L1 or anti-PD-1 antibody.

### BRIEF DESCRIPTION OF THE DRAWINGS

FIGS. 1A-1D. FIG. 1A. Schedule of immunotherapy. The tumor diameter was diagnosed by MRI imaging (bigger than 1 cm) and then 10 mg/kg of aPD-L1 was injected through systemic (IV). FIG. 1B. Tumor size changes after 14 days of systemic immunotherapy. FIG. 1C. Flow cytometry analysis of T cells (CD3+), MDSCs (CD11b/c+His48+) and Tregs (CD3+CD4+CD25+Foxp3+) from TILs of HCC tumor after IV injection of a PD-L1 (10 mg/kg) or non-treated control. FIG. 1D. H&E staining and TUNEL staining of HCC tumor slices after a PD-L1 immunotherapy. Arrows in the H&E panels indicate the non-nucleus necrosis, and arrows in the TUNEL panels indicate the TUNEL positive stained area. TUNEL positive area was calculated from histology section (shown in FIG. 14) by Qupath software. Data was obtained from different 3 independent samples. Data are shown as means±s.d. *p<0.05; **p<0.01; ***p<0.001, two-tailed paired t-test.

FIGS. 2A-2H. FIG. 2A. Strategy of aPD-L1 immobilization on NC (Fer) for the synthesis of aPD-L1-Z-Fer and aPD-L1@Fer. FIG. 2B. Schematic illustration of aPD-L1 orientation arranged Fer (aPD-L1-Z-Fer) by assembly of Fer-GSH and GST-Z, IgG Fc domain binding adaptor. FIG. 2C. GST-Z sequence (SEQ ID NO: 1) and process illustration of molecular cloning and protein purification of GST-Z. (i) Z inserts were amplified from Encapsulin-Z plasmid. GST-SC templates and Z inserts were digested by EcoRI and XhoI. Digested templates and inserts were purified and mixed to form the GST-Z plasmid. (ii) GST-Z plasmid was transformed to DH5α protein expression E. coli to produce the GST-Z protein. (iii) Immobilized metal affinity chromatography allows the purification of hexahistidine tagged GST-Z from supernatants of bacterial lysis solution. FIG. 2D. Electrophoresis data corresponding to the process of cloning and purification. (i) Agarose gel electrophoresis of PCR amplified Z inserts from Encapsulin-Z plasmid. (ii) Agarose gel electrophoresis of Z inserts and GST-SC plasmid templates (lane 2). After digestion with EcoRI and XhoI, templates (lane 4) and inserts (lane 3) were purified and mixed to form the GST-Z plasmid. (iii) SDS PAGE of eluted fractions from immobilized metal affinity chromatography. Most of GST-Z eluted in fractions 1 and 2 (lane 3, 4). FIG. 2E. Changes of hydrodynamic sizes by each step of synthesis. FIG. 2F. Non-reducing SDS PAGEs of aPD-L1-Z-Fer for the evaluation of aPD-L1 loading per Z-Fer (lane 7-13). Loaded aPD-L1 amount was quantified by standard curves (lane 1-5). FIG. 2G. Phantom MRI imaging to evaluate the T2 Contrast effect of aPD-L1-Z-Fer and aPD-L1@Fer. FIG. 2H. Calculation of R2 relaxivity (r2) based on the CNR (contrast to noise ratio). Data was obtained from at least different 3 independent samples.

FIGS. 3A-3E. FIG. 3A. Evaluation of the PD-L1 targeting affinity in physiological condition. PD-L1 positive adherent McA-Rh7777 cells in 10% FBS adjusted complete media with or without fluidics (0.5 mL/min) were applied to mimic the physiological condition. aPD-L1(AF488), aPD-L1(AF488)@Fer, and aPD-L1(AF488)-Z-Fer were slowly added, then evaluate the PD-L1 affinity by confocal laser scanning microscopy images. FIG. 3B. DSA assisted locoregional injection of aPD-L1-Z-Fer. To visualize the tumor, 2 mL of Omnipaque solution is applied before aPD-L1-Z-Fer injection. FIG. 3C. T2 MRI images of pre- or post-procedure locoregional IA delivery to NiS1 tumor. T2 contrast effect of Fer was utilized to visualize the tumor PD-L1 specific targeting. FIG. 3D. PD-L1 IHC and Prussian blue staining of tumor histology slices. Left bottom figure shows the dominant PD-L1 expression in tumor rim, and other figures are Prussian blue staining of N1S1 HCC tumor after indicated treatment. Orange arrows indicates the Prussian blue positive staining. FIG. 3E. Circulating aPD-L1 amount after locoregional delivery of aPD-L1@Fer and aPD-L1-Z-Fer. Circulating aPD-L1 amount was measured by ELISA from a serum sample. Data were obtained from at least different 3 independent samples. Data are shown as means±s.d. *p<0.05; **p<0.01; ***p<0.001, two-tailed paired t-test.

FIGS. 4A-4G. FIG. 4A. Scheduled timeline of in vivo experiment. After 7 days of NiS1 tumor inoculation, HCC tumor-bearing rats were treated with indicated therapy. Tumor size was traced at pre-, post-, 7-, and 14-days by 7T MRI. FIG. 4B. Enhanced contrast effect of aPD-L1@Fer and aPD-L1-Z-Fer assist the visualization diagnosis of HCC tumor. FIG. 4C. Relative tumor volume changes after indicated treatment. Tumor sizes were obtained from the largest tumor slices of MRI scanned images. FIG. 4D. Fold changes of tumor volume compared to day 14 and day 1. FIG. 4E. Tumor volume increase rate per day during the 14 days. FIG. 4F. H&E, TUNEL and CD3 staining of HCC tumor histology slices after indicated treatment. Arrows in H&E panels indicate necrosis of tumor. Arrows in TUNEL panels indicate the necrosis. Arrows in CD3 panels indicate the CD3 accumulation. FIG. 4G. Quantitative analysis of TUNEL positive and CD3 positive cells in HCC tumor. Data analyzed by Qupath software. Data were obtained from at least different 3 independent samples. Data are shown as means s.d. *p<0.05; **p<0.01; ***p<0.001, two-tailed paired t-test.

FIGS. 5A-5H. FIG. 5A. Changes of CD3+ TIL population after indicated treatment. Data obtained and analyzed by Flow cytometry. FIGS. 5B and C. Changes of MDSC (CD11b/c+His48+) and Treg (CD3+CD4+CD25+Foxp3+) population after indicated treatment. Data obtained and analyzed by Flow cytometry. FIG. 5D. Immunogenic conversion of TME increase the DC maturation (CD103+CD80+CD86+) after aPD-L1-Z-Fer treatment. Data obtained and analyzed by Flowcytometry. FIG. 5E. CD4 and CD8 T cell distribution changes of CD3+ T cell population from TILs. Data obtained and analyzed by Flow cytometry. FIG. 5F. Calculation of the changes of CD8 T cells to Tregs ratio in TIL population. FIG. 5G. Systemic inflammatory cytokines change after 14 days of indicated treatment, measured by IFN-γ, TNF cytokine CBA. FIG. 5H. Toxicity test based on the bodyweight changes. Data were obtained from at least different 3 independent samples. Data are shown as means±s.d. *p<0.05; **p<0.01; ***p<0.001, two-tailed paired t-test.

FIGS. 6A-6C. Molecular cloning and purification of GST-Z. FIG. 6A. Schematic illustration of GST-Z molecular cloning. Z sequence in Encapsulin-Z was amplified by PCR with EcoRI bearing forward primer and XhoI bearing reverse primer. Spycatcher sequence in GST-SC and amplified Z were then digested with restriction enzyme (EcoRI and XhoI) to make sticky ends. Finally, an intact form of GST-Z was synthesized by ligation of T4 ligase. FIG. 6B. PCR optimization of Z sequence with various annealing temperatures (50˜60 oC). DpnI restriction enzyme was added to PCR product to digest the methylated bacterial template plasmid, Encapuslin-Z in pET 21b. FIG. 6C. Solubility test prior to Ni-NTA affinity chromatography. 20 μL of samples from each step (IPTG induction, Bacterial cell lysis, Sonication) were collected and separated the supernatant (sup) and pellet (pt). SDS PAGE was processed to evaluate the GST-Z solubility.

FIGS. 7A-7C. Evaluation of GSH numbers on GSH-Fer and optimization of GST-Z binding to GSH-Fer. FIG. 7A. Schematic illustration of evaluation step of GSH number on GSH-Fer using maleimide-CF633. FIG. 7B. Standard curve of maleimide-CF633 absorbance to calculate the number of CF633, attached on the surface of Fer. Absorbance (O.D.) at 633 nm with different concentrations of maleimide-CF633 was analyzed by spectrophotometer, and the standard curve was obtained by linear fitting. Binding amount of CF633 was varied by initial concentration. 1:100 ratio of GSH-Fer to maleimide-CF633 showed the best binding efficiencies among the conditions. FIG. 7C. After GST-Z culture with GSH-Fer with molar ratio of 1:100, 1:150, 1:200. Excessive amount of GST-Z was removed by affinity exclusion with GSH-agarose. SDS PAGE data showed that the intensity of GST-Z monomer is almost similar between the ratios, which insist the saturation of GST-Z binding on GSH-Fer.

FIG. 8. ζ-potential changes of Fer-nanocarriers after formulation. After synthesis of GSH-Fer, Z-Fer, aPD-L1@Fer, and aPD-L1-Z-Fer, ζ-potentials were measured by Zetasizer Nano to evaluate the stability of colloidal dispersion in aqueous solution.

FIGS. 9A-9B. Quantitative analysis of aPD-L1 binding efficiency to Z-Fer. FIG. 9A. The standard curve of antibody concentration. To evaluate the amount of aPD-L1 after aPD-L1-Z-Fer synthesis, nonreducing SDS-PAGE was performed with various concentration of aPD-L1 (0.0625 to 40 M). Intensities of aPD-L1 were utilized to calculate the aPD-L1 amount of aPD-L1-Z-Fer based on the linear fitting formula of the aPD-L1 standard curve. FIG. 9B. aPD-L1 loading efficiency was evaluated by using input amount of aPD-L1 and output amount of aPD-L1. Loading efficiency was significantly decreased when aPD-L1 amount was higher than 1:7 ratio, Z-Fer to aPD-L1.

FIG. 10. In vitro PD-L1 expression of N1S1. To evaluate the amount of PD-L1 positive N1S1 cells, N1S1 cells were stained with PD-L1 antibodies. Flow cytometry analysis showed that around 8% of N1S1 cells expressed the PD-L1.

FIG. 11. Evaluation of equivalent loading of aPD-L1 to aPD-L1@Fer and aPD-L1-Z-Fer. After the formulation of aPD-L1@Fer and aPD-L1-Z-Fer, native PAGE was applied to quantify the equivalent loading of aPD-L1. Corresponding to 1.4 g aPD-L1 intensity of lane 2, aPD-L1@Fer and aPD-L1-Z-Fer showed similar intensity (land 9, 10), which supports the equivalent aPD-L1 loading.

FIG. 12. Enhanced MFI of aPD-L1-Z-Fer. aPD-L1(AF488), aPD-L1(AF488)@Fer, and aPD-L1(AF488)-Z-Fer were treated for 1 hours to evaluate the PD-L1 targeting affinity after preparation of N1S1 cells. MFI of aPD-L1(AF488) was quantified by flow cytometry. Data was obtained from at least 3 different independent samples.

FIG. 13. In vivo PD-L1 expression changes after treatment. After the treatment of aPD-L1 (IV), aPD-L1 (IV), aPD-L1@Fer, and aPD-L1-Z-Fer in an equivalent concentration of 10 mg/kg of aPD-L1, PD-L1 expression changes in the tumor were evaluated by flow cytometry. Compared to the 38% of initial PD-L1 positive tumor cell population in control, aPD-L1-Z-Fer showed a decrease of 16% in PD-L1 positive tumor cells.

FIG. 14. Low magnification images of representative H&E, TUNEL and CD3 histology slices for quantitative analysis.

FIG. 15. Liver function test of blood serum chemistry after treatments. Amounts of alanine aminotransferase (ALT), aspartate aminotransferase (AST), creatinine, albumin, bilirubin unconjugated, gamma-glutamyl transferase, and total protein were quantified at two weeks following treatments with PBS (Control), aPD-L1 (IV), aPD-L1 (IA), aPD-L1@Fer, and aPD-L1-Z-Fer. There was no statistical significance between the groups by one-way ANOVA. Data are presented as the mean±SEM (n=3).

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

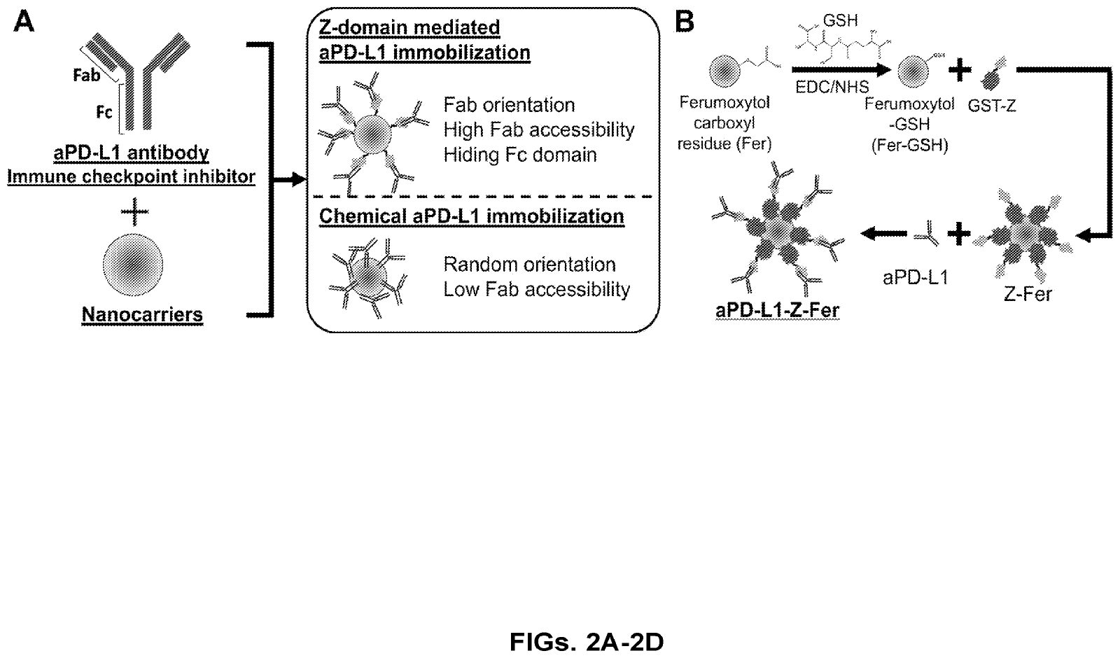

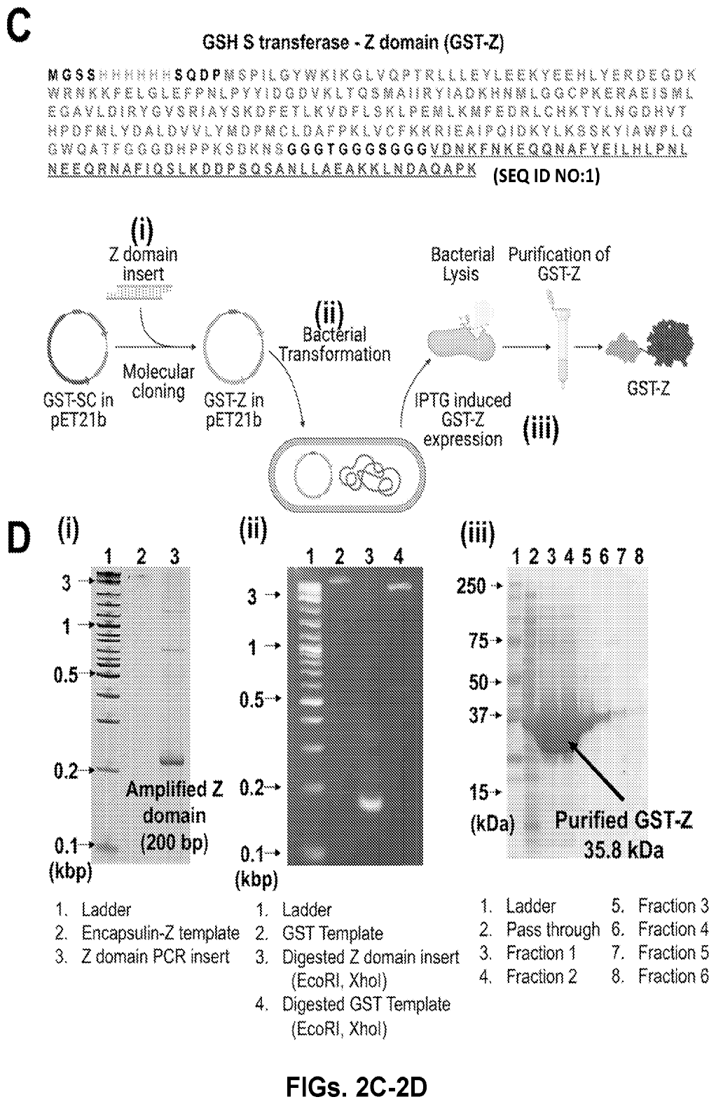

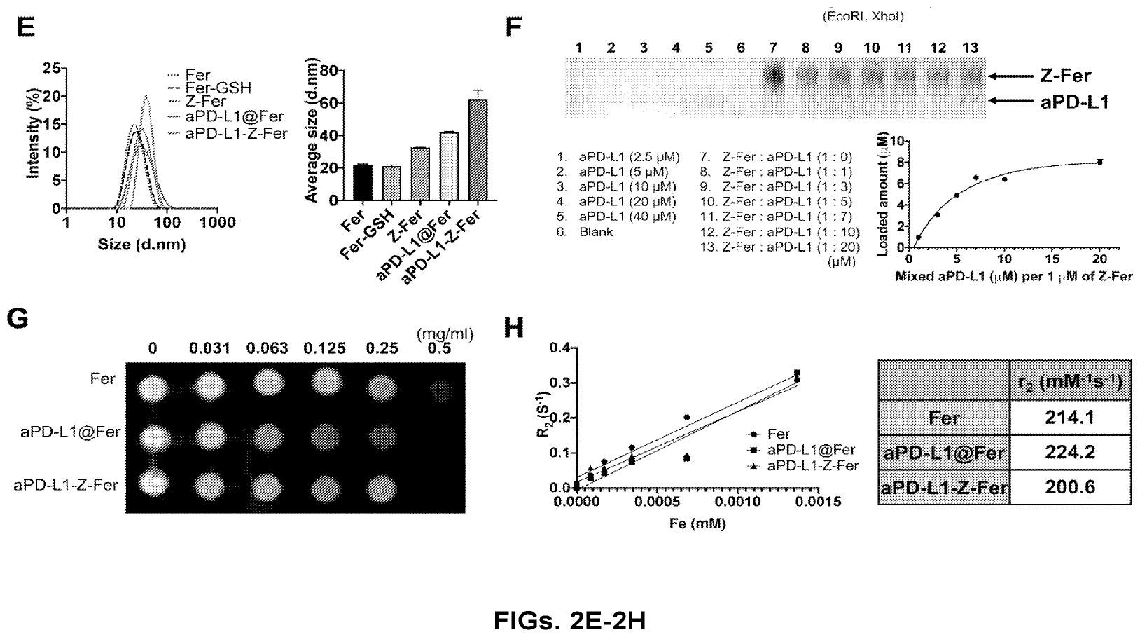

The present disclosure describes a new immune checkpoint antibody formulation having oriented immune checkpoint antibodies directionally attached to iron oxide nanoparticles which provide improved properties for delivery and treatment of immune checkpoint (IC) therapy. As demonstrated in the examples, an anti-PD-L1 mAb (aPD-L1) was directionally conjugated on iron oxide nanoparticles for local immunotherapy of cancer. An engineered Z-domain (Z), which is an immunoglobulin G (IgG) Fc-specific binding protein, was incubated and conjugated to iron oxide nanoparticles (e.g., Ferumoxytol (Fer)). This allowed for control of the orientation of binding of the immune checkpoint antibodies to the iron nanoparticles. This directional binding allowed for the Fab binding portion of the antibodies to be facing out from the nanoparticles, allowing for coating of the nanoparticles with a layer of active binding domain (Fab) on the surface as depicted in FIG. 2B. As demonstrated in the examples, the engineered aPD-L1-Z-Fer ICI formulation demonstrates enhanced affinity and avidity for effective immune checkpoint blockade of PD-L1 expressed HCC cells in vitro. Transcatheter-directed hepatic intra-arterial (IA) local aPD-L1-Z-Fer ICI immunotherapy showed superior in vivo HCC tumor suppression and immune conversion compared to systemic aPD-L1 alone or chemically conjugated aPD-L1-Fer. Thus, the developed aPD-L1-Z-Fer provides a new local HCC immunotherapy with improved tumor suppression and immune activation.

The inventors formulated immune checkpoint inhibitor decorated iron oxide nanoparticles for high efficient immune checkpoint inhibitor cancer therapy. To formulate the ICI formulations of the present invention, each functionalized Fer and Z-domain were combined via GSH-GST ligand-receptor attachment (Z-Fer). Then, a simple co-incubation of the immune checkpoint inhibitor antibody (e.g., aPD-L1) with the Z-Fer allowed the preferential connection between the Fc region of the antibody (aPD-L1) and the Z on Fer, forming aPD-L1-Z-Fer having specific directionality. GST functionalized Z protein (GST-Z) was engineered as shown in the protein sequence (SEQ ID NO: 1) and FIG. 2C. Z sequence was genetically introduced to the posterior position of GST by molecular cloning. GST-Z protein was overexpressed by E coli (DH5a) with IPTG (isopropyl ß-D-1-thiogalactopyranoside) induction. Then, it was purified with immobilized metal affinity chromatography. The surface of Fer was modified with GSH, to produce GSH-Fer. The complete form of Z-Fer was assembled by co-incubation of GST-Z and GSH-Fer. Engineered Z-Fer spontaneously immobilized the antibody (e.g., aPD-L1 ICI mAb) to form an antibody-nanoparticle complex (aPD-L1-Z-Fer) by a simple co-incubation step.

Compositions and methods of treatment using locoregional compound delivery are disclosed herein. It is to be understood that the disclosed compositions, methods of use, and methods of preparing said compositions are intended for use in any procedure where locoregional delivery of a compound or compounds, particularly immunotherapy, is desired or intended. Therefore, the disclosed compositions, methods of use and methods of preparing compositions for use in locoregional delivery are intended for use in, by way of example but not by way of limitation, intratumoral administration, intrahepatic administration, transarterial administration, or other locoregional delivery routes or procedures.

In a first aspect of the invention, iron nanoparticles comprising directionally attached antibodies are provided. In some embodiments, the nanoparticles comprise an immune checkpoint inhibitor (ICI) antibody directionally attached by the constant region (Fc) of the antibody to an iron nanoparticle with a directional linker, wherein the antigen binding sites of the antibody are facing outwardly from the nanoparticle. In some embodiments, the directional linker is a Z-domain. The Z-domain is linked to the nanoparticle through GST-glutathione linkage, e.g., the nanoparticle is coated with glutathione and the Z-domain is a fusion protein comprising the Z-domain directionally tethered to GST protein. FIG. 2B illustrates the nanoparticles described herein.

As used herein, “nanoparticle” refers to a particle of matter which has a diameter of less than 1000 nm. As used herein, “iron nanoparticle” refers to a nanoparticle comprising iron. In some embodiments, iron nanoparticles comprise particles of Fe₃O₄ also known as “Iron (II, III) oxide”, or “magnetite”. In some embodiments, iron nanoparticles comprise chemical modifications on their surfaces. In some embodiments, the surface of the iron nanoparticles comprises free carboxyl groups. In some embodiments, glutathione is conjugated to the surface of the nanoparticles.

As used herein, “directional linker” refers to a peptide, nucleic acid, or combination thereof that is capable of attaching an antibody to a nanoparticle by the Fc region, or trunk of the Y-shaped antibody, such that the antigen binding region, or variable region, is exposed and facing roughly perpendicularly to the surface of the nanoparticle. In some embodiments, the directional linker is a glutathione S transferase (GST) molecule linked to the Z domain, or Fc antibody binding domain, of staphylococcal protein A. In some embodiments, the directional linker includes a sequence that allows for efficient purification of the linker when the linker is produced in a genetically engineered cell and isolated, for example, a poly His sequence. In some embodiments, the directional linker has the sequence: M G S S H H H H H H S Q D P M S P I L G Y W K I K G L V Q P T R L L L E Y L E E K Y E E H L Y E R D E G D K W R N K K F E L G L E F P N L P Y Y I D G D V K L T Q S M A I I R Y I A D K H N M L G G C P K E R A E I S M L E G A V L D I R Y G V S R I A Y S K D F E T L K V D F L S K L P E M L K M F E D R L C H K T Y L N G D H V T H P D F M L Y D A L D V V L Y M D P M C L D A F P K L V C F K K R I E A I P Q I D K Y L K S S K Y I A W P L Q G W Q A T F G G G D H P P K S D K N S G G G T G G G S G G G V D N K F N K E Q Q N A F Y E I L H L P N L N E E Q R N A F I Q S L K D D P S Q S A N L L A E A K K L N D A Q A P K (SEQ ID NO: 1). In some embodiments, GST has the sequence: M S P I L G Y W K I K G L V Q P T R L L L E Y L E E K Y E E H L Y E R D E G D K W R N K K F E L G L E F P N L P Y Y I D G D V K L T Q S M A I I R Y I A D K H N M L G G C P K E R A E I S M L E G A V L D I R Y G V S R I A Y S K D F E T L K V D F L S K L P E M L K M F E D R L C H K T Y L N G D H V T H P D F M L Y D A L D V V L Y M D P M C L D A F P K L V C F K K R I E A I P Q I D K Y L K S S K Y I A W P L Q G W Q A T F G G G D H P P K S D K N S (SEQ ID NO: 2). In some embodiments, Z domain has the sequence: V D N K F N K E Q Q N A F Y E I L H L P N L N E E Q R N A F I Q S L K D D P S Q S A N L L A E A K K L N D A Q A P K (SEQ ID NO: 3).

As used herein, “glutathione” or “GSH” refers to a tripeptide with a gamma peptide linkage between the carboxyl group of the glutamate side chain and cysteine. The carboxyl group of the cysteine residue is attached by normal peptide linkage to glycine. GSH is a substrate of the enzyme glutathione S-transferase.

As used herein, “glutathione S-transferase” or “GST” refers to a class of enzymes which catalyze the conjugation of the reduced form of glutathione (GSH) to substrates. GST binds tightly to glutathione (GSH) and, thus, is used to attach compounds or molecules bound to GSH to compounds or molecules bound to GST. In some embodiments, nanoparticles have GSH bound to their surface and are contacted to GST bound to an antibody thereby linking the antibody to the nanoparticle. In further embodiments, the GST is attached to a Z domain which directionally binds the Fc region of the antibody thereby imparting directionality to the directional linker comprising GST and the Z domain to the nanoparticle to which it is tethered.

As used herein, “Z domain” refers to the Z domain of staphylococcal protein A. The Z domain is homologous to the five individually folded E, D, A, B and C domains of protein A and it was derived by introducing a chemically stabilizing mutation in the B domain. See Nilsson et al. Protein Engineering, Design and Selection, Volume 1, Issue 2, February 1987, Pages 107-113. Critically, the Z domain binds antibodies on the Fc domain.

As used herein, “tumor microenvironment” refers to the environment around a tumor, including the surrounding blood vessels, immune cells, fibroblasts, signaling molecules and the extracellular matrix (ECM).

In some embodiments, the compositions and methods disclosed herein reduce immune-related adverse events (irAEs) when compared to ICIs alone. irAEs are defined as a unique spectrum of side effects of ICIs that resemble autoimmune responses. irAEs affect almost every organ of the body and are most commonly observed in the skin, gastrointestinal tract, lung, and endocrine, musculoskeletal, and other systems. Therefore, compositions and methods that lead to a reduction of irAEs represent important innovations in the field.

As used herein, “immunotherapeutics,” “immunotherapies” or “immune-boosting agents” refers to molecules, chemicals and compounds that elicit an immune response and include, for example, checkpoint inhibitors, cancer vaccines, adoptive cell transfer therapies (ACT), and small molecules, among others. As used herein, these immune boosting agents refers to agents, molecules, and compounds that induce an increase the intensity, effectiveness, or duration of an immune response. Exemplary immune-boosting agents include anti-PD-1 monoclonal antibodies, anti-PD-L1 monoclonal antibodies, anti-CTLA-4 monoclonal antibodies, anti-VISTA monoclonal antibodies, or other compounds targeting “immune checkpoint” molecules.

As used herein, “immune checkpoints” refers to proteins or peptides that regulate the activity of an immune response. For example, some immune checkpoints interfere with the ability of the immune system to mount an effective response. By way of example but not by way of limitation, immune checkpoints include the PD-1:PD-L1/PD-L2 axis.

As used herein, “immune checkpoint therapy” (“ICT”) refers to an intervention that is targeted to interfere with the normal function of “immune checkpoints.” In some embodiments, ICT comprises a treatment that interferes with the function of PD-1 or its ligands PD-L1 and PD-L2. In some embodiments, the ICT comprises a monoclonal antibody targeted to PD-1. In another example, the ICT is a monoclonal antibody targeting PD-L1. In some embodiments, the monoclonal ICT therapy is selected from the group consisting of pembrolizumab, nivolumab, cemiplimab, atezolizumab, dostarlimab, durvalimab, and avelumab.

Checkpoint inhibitors that comprise anti-PD1 antibodies or anti-PDL1-antibodies or fragments thereof are known to those skilled in the art, and include, but are not limited to, cemiplimab, nivolumab, pembrolizumab, MEDI0680 (AMP-514), spartalizumab, camrelizumab, sintilimab, toripalimab, dostarlimab, and AMP-224. Checkpoint inhibitors that comprise anti-PD-L1 antibodies known to those skilled in the art include, but are not limited to, atezolizumab, avelumab, durvalumab, and KN035. The antibody may comprise a monoclonal antibody (mAb), chimeric antibody, antibody fragment, single chain, or other antibody variant construct, as known to those skilled in the art. PD-1 inhibitors may include, but are not limited to, for example, PD-1 and PD-L1 antibodies or fragments thereof, including, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83,545,509, 8,900,587 and EP2170959; atezolizumab is an anti-PD-L1 available from Genentech, Inc. (Roche) and described in U.S. Pat. No. 8,217,149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi, see, e.g., U.S. Pat. Nos. 9,938,345 and 9,987,500), spartalizumab (PDR001, Novartis), camrelizumab (AiRuiKa, Hengrui Medicine Co.), sintillimab (Tyvyt, Innovent Biologics/Eli Lilly), KN035 (Envafolimab, Tracon Pharmaceuticals, see, e.g., WO2017020801A1); tislelizumab available from BeiGene and described in U.S. Pat. No. 8,735,553; among others and the like. Other PD-1 and PD-L1 antibodies that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others (the references citations regarding the antibodies noted above are incorporated by reference in their entireties with respect to the antibodies, their structure and sequences). Fragments of PD-1 or PD-L1 antibodies include those fragments of the antibodies that retain their function in binding PD-1 or PD-L1 as known in the art, for example, as described in AU2008266951 and Nigam et al. “Development of high affinity engineered antibody fragments targeting PD-L1 for immunoPED,” J Nucl Med May 1, 2018 vol. 59 no. supplement 1 1101, the contents of which are incorporated by reference in their entireties.

As used herein, “dendritic cell (DC) maturation” involves a redistribution of major histocompatibility complex (MHC) molecules from intracellular endocytic compartments to the DC surface, down-regulation of antigen internalization, an increase in the surface expression of costimulatory molecules, for example CD80 and/or CD86, morphological changes (e.g. formation of dendrites), cytoskeleton re-organization, secretion of chemokines, cytokines and proteases, and surface expression of adhesion molecules and chemokine receptors.

As used herein, “chemotherapeutics” refers to compounds used to treat cancer including, but not limited to, cytotoxic agents, targeted therapies, and hormonal therapies. Exemplary chemotherapeutics for use in the compositions and methods of the current disclosure include: actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilon, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine. In some embodiments, exemplary chemotherapeutics for use in the compositions and methods of the current disclosure are daunorubicin, doxorubicin, epirubicin, idarubicin, valrubicin, Sorafenib, regorafenib, and Lenvatinib.

As used herein, “locoregionally” refers to the condition of being limited to a local region of a subject's body. Locoregionally includes, for example, for tumor therapy, in the region in which the tumor is located (e.g., organ or surrounding area).

As used herein, “Fc” or “Fc region” refers to the fragment crystallizable region of the antibody. It consists of the tail region of the antibody consisting of the two identical protein fragments derived from the second and third constant domains of the antibody's two heavy chains, i.e., four total constant regions per complete antibody structure that bind to Fc receptors (FcR) on immune cells.

As used herein, “binding affinity” or simply “affinity” refers to the strength of binding of a single molecule to its ligand. It is typically measured and reported by the equilibrium dissociation constant (K_{D}), which is used to evaluate and rank order strengths of bimolecular interactions.

As used herein, “resistance to fluidic shear force” refers to the property of a molecule, e.g., and antibody, which when bound to a ligand, is bound tightly such that shear forces generated by a fluid moving across the surface to which the antibody is bound do not tear the antibody away from its ligand. Put another way, resistance to fluidic shear force is associated with the property of a molecule to stay tightly bound to its ligand. In some embodiments, the compositions of the current disclosure possess resistance to shear forces such that the shear forces generated by blood flowing through the circulatory system do not remove the compositions from their target ligands.

In a second aspect of the invention, pharmaceutical compositions comprising the nanoparticles are provided.

In a third aspect of the invention, methods of treating a subject in need of treatment for cancer are provided. In some embodiments, the methods comprise administering an effective amount of a composition comprising an antibody directionally attached to an iron nanoparticle with a directional linker, wherein the antigen binding sites are facing outwardly from the nanoparticle to treat the cancer. In preferred embodiments, the antibody is an immune checkpoint inhibitor (ICI) antibody.

In some embodiments, the nanoparticles having a directionally attached antibody disclosed herein have a greater binding affinity for an antigen and an increased resistance to fluidic shear force than the antibody alone or a nanoparticle having an antibody chemically linked directly to the nanoparticle. In some embodiments, the nanoparticles disclosed herein induce increased CD3+ cell accumulation in a tumor microenvironment (TME), reduce myeloid derived suppressor cell (MDSC) accumulation in a TME, and/or increase dendritic cell (DC) maturation compared to the antibody alone or a nanoparticle having the antibody chemically linked directly to the second nanoparticle. “Chemically linked directly” means that there is no intervening linker between the nanoparticle and the antibody.

As used herein, the terms “administering” and “administration” refer to any method of providing a pharmaceutical preparation to a subject. Such methods are well known to those skilled in the art and include, but are not limited to, transarterial administration, intra-arterial administration, intrahepatic administration, and intratumoral administration. Administration can be continuous or intermittent. In various aspects, a preparation can be administered therapeutically; that is, administered to treat an existing disease or condition.

As used herein, “cancer” or “tumor” refers to diseases, e.g., cell proliferative diseases, wherein an organism's cells grow uncontrollably and may spread to other locations in the organism (e.g., metastasize). By way of example, any cancer currently or that may be treated at some point with TACE may be contemplated by the present invention. Suitable cancers include, but are not limited to, hepatoma, hepatocellular carcinoma (primary liver cancer), cholangiocarcinoma (primary cancer of the bile ducts in the liver), metastasis in the liver from other cancers, including, for example, colon cancer, breast cancer, carcinoid tumors, neuroendocrine tumors, islet cell tumors of pancreas, ocular melanoma, vascular primary tumors, among others. In some embodiments, cancer refers to hepatocellular carcinoma (HCC).

As used herein, “transarterial chemoembolization (TACE)” or “transarterial embolization” refers to an image-guided, non-surgical procedure that is used to treat malignant lesions in the liver. The procedure uses an X-ray guided catheter to deliver both chemotherapy medication and embolization materials into the blood vessels that lead to the liver and to the tumor.

For purposes of the present invention, “treating” or “treatment” describes the management and care of a subject for the purpose of combating the disease, condition, or disorder. Treating includes the administration of a compositions described herein when it is determined that the subject would be provided a benefit by the administration of the treatment to prevent the onset of the symptoms or complications, alleviating the symptoms or complications, or eliminating the disease, condition, or disorder.

The term “treating” can be characterized by one or more of the following: (a) reducing, slowing or inhibiting the growth of cancer, including reducing slowing or inhibiting the growth of cancer cells; (b) preventing the further growth of tumors; (c) reducing or preventing the metastasis of cancer within a patient, and (d) reducing or ameliorating at least one symptom of the cancer. In some embodiments, the optimum effective amounts can be readily determined by one of ordinary skill in the art using routine experimentation.

A “subject in need thereof” as utilized herein may refer to a subject in need of treatment for a disease or disorder characterized by a tumor that is treatable by locoregional delivery of an immunotherpeutic agent. A subject in need thereof may include a subject suffering from hepatocellular carcinoma (HCC). A subject in need thereof may include a subject in need of treatment by transarterial embolization. A subject in need thereof may include a subject in need of transarterial chemoembolization (TACE). A subject in need thereof may include a subject having a cancer for which treatment of the cancer would benefit from immune stimulation, especially local immune stimulation.

As used herein, the terms “effective amount” and “therapeutically effective amount” refer to the quantity of active therapeutic agent or agents sufficient to yield a desired therapeutic response without undue adverse side effects such as toxicity, irritation, or allergic response. The specific “effective amount” will, obviously, vary with such factors as the particular condition being treated, the physical condition of the subject, the type of animal being treated, the duration of the treatment, the nature of concurrent therapy (if any), and the specific formulations employed and the structure of the compounds or its derivatives.

As used herein, “CD3⁺ cells” refers to cells that stain positively by flow cytometry, immunofluorescence, immunohistochemistry, or electron microscopy for an antibody directed against CD3. It is to be understood that CD3 consists of a family of proteins which are indicative of T cells including CD8⁺ T cells, CD4⁺ T cells, and γδ T cells. Therefore, infiltration of CD3⁺ cells into the tumor microenvironment is associated with an increase in inflammatory anti-tumor immune response.

As used herein, “myeloid derived suppressor cells” or “MDSCs” refers to myeloid cells with the capacity to suppress an inflammatory immune response or to directly suppress T cell responses. In mice, MDSCs may be characterized as CD11b⁺, Ly6C⁺, Ly6G⁻ or CD11b⁺, Ly6C⁺, Ly6G⁺, or by other markers known in the art. In humans, MDSCs may be characterized as CD33⁺, CD14⁺, CD15⁻ or CD33⁺, CD14⁻, CD15⁺, or by other markers known in the art. In rats, MDSCs may be characterized as CD11b/c⁺ and His48⁺, or by other markers known in the art.

As used herein, “innate immune response” refers to an innate immune interaction with tumor cells, which can include but is not limited to, for example, recognition by innate cell populations (NK cells, NKT cells, and γδ T cells) and also by dendritic cells and macrophages, CD8+ T cell responses and other pathways that stimulate the innate immune system, e.g., therapeutic stimulation of pattern recognition receptors (PRRs), such as Toll-like receptors (TLRs); the DNA sensing cGAS/STING pathway; nucleotide-binding oligomerization domain-like receptors (NLRs), such as NLRP3; and the retinoic acid-inducible gene-I (RIG-I)-like receptors (RLRs). Nonspecific and immediate immune responses are classified as innate due to their fast-acting nonspecific response against foreign antigens. Adaptive immune response involves the development of immunological memory due to specific forms of immune responses targeting the antigens with naïve lymphocytes, such as the T and B cells, gaining the ability to differentiate and mature into either effector T cells (CD4+ or CD8+ T cells) or antibody-secreting B cells (plasma cells), and is well understood in the art. CD4⁺ T cells CD4⁺ T cells can differentiate into several subsets of effector T cells such as T helper 1 (Th1) cells, T helper 2 (Th2) cells, or Tregs, with each of these subsets of CD4⁺ T effector cells can produce and secrete certain cytokines that modulate immune response accordingly. Similar to NK cells in innate immunity, naïve CD8⁺ T cells rely on MHC class I for maturation into effector cytotoxic T cells. CD8⁺ T cells via the specific T cell receptor bind to the antigen/MHC class I complexes on the antigen-presenting cells (i.e., target cells) resulting in release of perforin and granzymes from CD8⁺ T cells and death of the target cell. Methods of determining the activation of an innate or adaptive immune response are well known in the art.

As demonstrated in the examples, formulated aPD-L1-Z-Fer of the present invention showed a strong MRI T2 contrast effect due to the Fer core. HCC tumor size changes after 14 days of DSA assisted locoregional injection of aPD-L1-Z-Fer are demonstrated showing a significant reduction in tumor size and volume. The ratio of tumor killing CD8 T cell population and immune suppressive regulatory T cells (Tregs) was obtained by flow cytometry analysis was also demonstrated.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following Examples are illustrative and should not be interpreted to limit the scope of the claimed subject matter.

### Example 1: Design and Testing of Directionally Loaded Nanoparticles with ICI

This example demonstrates that directionally oriented nanoparticles increased the local delivery and efficacy of the immune checkpoint inhibitor (ICI) and reduced toxic side effects of the composition when used to treat cancer. Further, this example demonstrates formulations and methods of activating an immune response to tumors by administration of the directionally oriented nanoparticles linked to ICI antibodies.

HCC is the 5^{th} most common malignancy in the world.¹ HCC incidence is rising, and it is projected to increase over the next two decades.² Most systemic and regional therapies offer palliation rather than cure. Systemic chemotherapy and hormonal therapy offer limited survival benefit^{3, 4}; and regional minimally invasive therapies, including thermal and chemical ablation, have limited efficacy with frequent recurrence.^{5, 6} Other treatment options include catheter-directed therapies, such as transcatheter arterial embolization (TAE), chemoembolization (TACE), and ⁹⁰Y-radioembolization (⁹⁰Y-RE). These transcatheter therapies take advantage of the fact that liver tumors derive their blood supply mainly from the hepatic artery, whereas normal liver tissues receive blood via the portal system.⁷ Catheter directed therapies improve liver cancer patient survival but the overall prognosis of these patients remains modest.^{8, 9} As demonstrated by promising immuno-therapeutic outcomes in melanoma, lung cancer and renal cell carcinoma,¹⁰ immune checkpoint inhibitors (ICIs) immunotherapy has emerged as an effective and promising treatment for HCC.^{11, 12} However, recent phase I/II trials of ICI immunotherapy for HCC patients showed a moderate response rate of 19%.^{12, 13} Current systemic administration of ICI immunotherapy injecting anti-CTLA4 (aCTLA4), anti-PD-1 (aPD-1) or anti-PD-L1 (aPD-L1) immunoglobulin G (IgG) based monoclonal antibodies (mAbs) may not be effective to achieve an anti-cancer immune response in immune-suppressive and hypoxic HCC.^{14, 15, 16} Systemic non-specific exposure, opsonization and early elimination of ICI mAbs are the critical hurdles, which are relevant to therapeutic efficacy (overall survival (OS), progression-free survival (PFS), response rates (RRs) and immune-related adverse events (irAEs)).¹⁷ PD-1/PD-L1 ICI mAbs are mostly humanized or human IgG antibodies displaying approximately the same pharmacokinetics as other therapeutic mAbs in the systemic administration. Intravenously (IV) administered ICI mAbs are directly circulated in the central vasculature and then distributed to peripheral tissues near the tumors. During the circulation, extensive off-target binding and proteolytic clearance in the plasma or peripheral tissues, along with non-specific ICI mAb binding mediated irAEs, reduce the delivery amount of ICIs mAbs to the targeted tumor.¹⁸ IC molecules expressed in normal tissues of the periphery induce the target mediated drug disposition and off-target irAEs, limiting the allowable administered doses.^{17, 19} Thus, effective delivery of ICI mAbs is crucial for the treatment of immune-suppressive tumors such as HCC. For enhancing targeted affinity of IV injected ICI mAbs during circulation, various genetic and structural modifications of mAb have been studied.¹⁷ However, the ICI delivery and off-target mediated irAEs are still challenging for ICI immunotherapy.

Recently, the importance of nanocarriers (NCs) has been highlighted for ICI mAb delivery.^{20, 21, 22} Various antibody conjugated NC platforms have been studied as “nano-immunotherapies”and showed enormous potential to improve the efficacy of ICI immunotherapy.^{23, 24, 25} However, non-controlled IgG-based ICI mAb conjugation on the surface of NCs often hides the IC binding Fab domain and exposes the FcγR binding domain of ICI mAb, causes low IC binding affinity and rapid clearance with FcγR-mediated endocytosis and protein adsorption of the unstable ICIs-NCs conjugates.^{26, 27, 28, 29} In this regard, recent studies proved that the proper direction of IgG mAbs onto NCs yields more affinity and specificity than randomly oriented IgG mAbs in antibody-nanoconjugates.³⁰ Intactly arranged outward Fab of mAb on the NCs achieves the maximal affinity to the target tumor.³¹ The steric hindrance and uniform surface of arranged mAbs also reduce the interaction with serum proteins to enhance the targeted binding efficiency.³²

Herein Z-domain (Z), an IgG Fc domain-specific binding protein from Staphylococcus aureus protein A,^{33, 34} was engineered to control the oriented conjugation of aPD-L1 mAbs ICI with FDA approved magnetic nanoparticles (ferumoxytol; Fer) for effective ICI mAbs delivery. Engineered GST-Z adaptors, which preferentially bind with the Fc domain of aPD-L1, were attached to Fer. A simple co-incubation of the synthesized Z-Fer and aPD-L1 readily allowed oriented immobilization of aPD-L1 that forms facing-out Fab and cloaking Fc domain of aPD-L1 on Z-Fer.^{35, 36} Enhanced affinity and avidity of engineered Z mediated aPD-L1 nanoconjugates (aPD-L1-Z-Fer) in blocking the PD-1/PD-L1 axis were evaluated in vitro and in vivo compared to only aPD-L1 or aPD-L1 covalently conjugated with Fer (aPD-L1@Fer). Based on previous findings^{37, 38, 39, 40}, tumor-specific local delivery of ICI mAb loaded NCs maximize ICI dose and IC blocking efficacy, resulting in enhanced anti-cancer immune response with minimized irAEs. Recently, the administration route of ICI has received greater attention, and increasing evidence confirms that comparable efficacy may be achieved by local intratumoral or tumor-draining lymph nodes administration of immunotherapies with better tolerability.^{41, 42, 43} Various clinical practices requiring HCC specific delivery have utilized the transcatheter-directed intra-arterial (IA) infusion as a standard.^{44, 45, 46, 47} Transcatheter-directed hepatic IA infusion was adopted for evaluating in vivo therapeutic potential of local delivered aPD-L1-Z-Fer in HCC. In an HCC rat model, aPD-L1-Z-Fer was delivered to HCC via transcatheter-directed IA infusion. Enhanced in vivo HCC tumor suppression converted immune-suppressive TME, and minimized irAEs by effective immune checkpoint blockade of IA delivered aPD-L1-Z-Fer were evaluated for an advanced local immunotherapeutic approach of HCC.

### Results

The inferiority of ICI immunotherapy in HCC compared to other solid tumors is mainly attributed by tumor resistance or ignorance with the unique, strong immune suppression and hypoxia. During the development of HCC, chronic inflammation accompanied by liver cirrhosis promotes the IC overexpression, infiltration of immune-suppressive regulatory T cells (Tregs), and myeloid-derived suppressor cells (MDSCs) in the tumor microenvironment (TME).^{48, 49, 50} HCC hypoxia-mediated overexpression of hypoxia-inducible factor 1□ (HIF-1 □) also activates the VEGF/R associated neo-angiogenesis and CXCL12 secretion, inducing immune-suppressive cells infiltration.^{51, 52} The physiological barriers, including the abnormal heterogeneous vasculature of HCC, also hinders the therapeutic efficacy of ICI immunotherapy.^{53, 54, 55} Current clinical trials for immunotherapy of HCC are mostly focusing on the systemic ICI combination with the first line Sorafenib treatment. VEGF/R inhibitor Sorafenib provides less hypoxic TME for immunotherapy. The addition of ICIs was expected to show robust immune responses to cancer cells.⁵⁶ However, a clinical trial of Sorafenib with Pembrolizumab combination therapy (IV, 200 mg every 3 weeks) showed no significant OS and PFS benefits in HCC (Keynote-240, NCT02702401). Other clinical trials using systemic administration of Ipilimumab and Nivolumab combination therapy after Sorafenib treatment (IV, 4 doses Nivolumab 1 mg/kg plus Ipilimumab 3 mg/kg every 3 weeks then Nivolumab 240 mg every 2 weeks) showed only a slight increase of OS with 94% of irAEs (Checkmate-040, NCT01658878). Systemic IV delivered ICIs might not be enough to activate the cancer-specific immunity in the highly immune-suppressive TME of HCC.

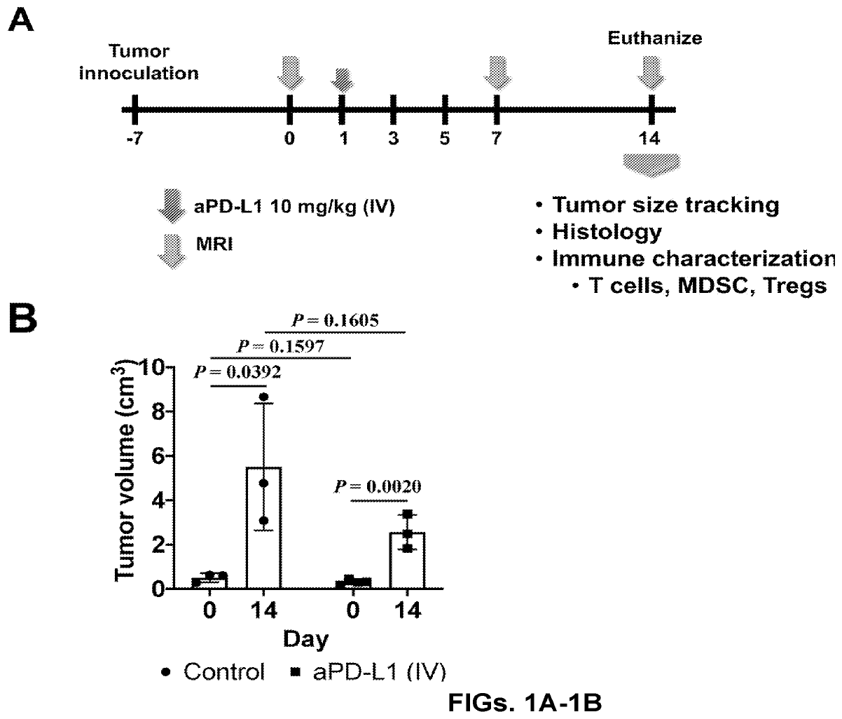

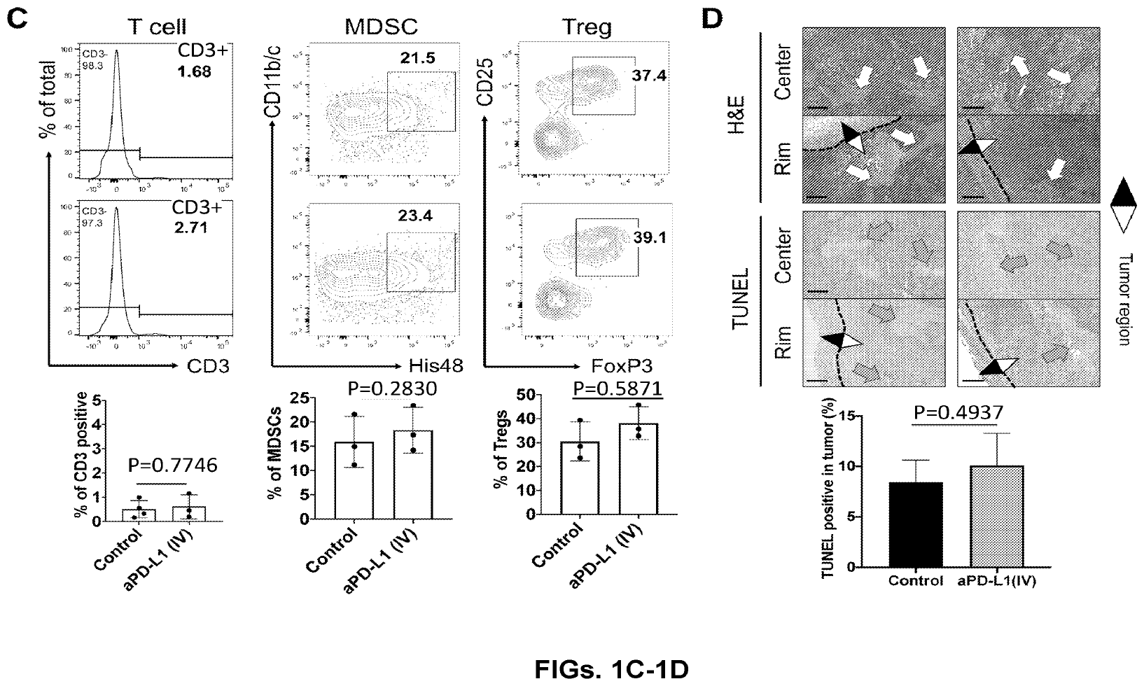

Although pre-clinical HCC rat models are frequently used in interventional oncology research, immune-suppressive TME of HCC has not been reported well. Here, highly immune-suppressive TME and the challenge of systemic delivered ICI immunotherapy in HCC could be observed in the syngeneic NS HCC rat model. A maximum clinical dose of aPD-L1 (10 mg/kg)⁵⁷ was administered to orthotopic N1S1 HCC rats via IV (FIG. 1A). After 14 days of the treatment, the tumor growth and immune changes were characterized compared to non-treated control N1S1 rats. The tumor size changes after the IV injection of aPD-L1 were not significantly different with the non-treated control group (FIG. 1). Only a small number of CD3 positive T cell population was infiltrated in the tumor treated with IV injection of aPD-L1 as similar level with non-treated control (FIG. 1C). High amounts of immune-suppressive cells including MDSC (21.5%) and Tregs (37.4%) of non-treated HCC were not changed after IV injection of aPD-L1 (FIG. 1C). Histology images and quantitative analysis of tumor sections stained with hematoxylin and eosin (H&E) and terminal deoxynucleotidyl transferase deoxyuridine triphosphate (dUTP) nick end labeling (TUNEL) also showed no significant difference of cancer cell death between the groups of IV injection of aPD-L1 and control (FIG. 1D), as shown in the tumor progression for 2-week post treatment (FIG. 1). One maximum dose of IV injected aPD-L1 used in the clinic might not be effective to change the immune-suppressive TME of HCC for suppressing tumor growth. Due to the immune-suppressive HCC TME presenting high amount of MDSC and Tregs, aPD-L1 dosages are generally high to modulate TME in the clinic.⁵⁸ However, PD-L1 is expressed in both tumor cells and various immune cells of the body.^{59, 60} IV injected aPD-L1 might results in low immune checkpoint blockage in targeted tumor region by non-specific binding with systemic proteins, tissues and cells. Thus, overall therapeutic outcomes are remained in moderate or low level. Although this is a limited study with a maximum clinical dosage of aPD-L1, in vivo demonstration of immune suppressive TME and immune/tumor responses after systemic IV injected aPD-L1 in HCC rats provides a feasibility of testing the local immunotherapeutic strategy using IA delivered aPD-L1-Z-Fer and helpful information for investigating various immunotherapies of HCC in the preclinical stage.

### Engineering Z-Domain and Z-Domain Functionalization of Ferumoxytol

NCs, which deliver multiple ICI mAbs in a single unit, are one of the promising options to achieve enhanced IC blockade.^{19, 61} The inventors synthesized oriented aPD-L1 conjugated Fer with Z adaptors for maximizing PD-L1 blocking efficiency for the treatment of HCC. In the nano-immunotherapy area, ICI mAbs are commonly loaded into NCs by chemical immobilization. Chemical immobilization methods often choose various crosslinkers connecting amine groups or thiol groups of ICI mAb with designated functional groups of NCs. However, both approaches result in randomly oriented ICI mAb conjugation that exhibits cloaked target-specific Fab region and exposed Fc domains of ICI mAb on NCs (FIG. 2A). Well-oriented ICI mAb conjugation with NCs is critical to enhance the ICI binding and ICI immuno-therapeutic efficacy.³⁰ In this experiment, Z of protein A, which has a superior affinity to the Fc domain of IgG mAb, was engineered for aPD-L1 mAb immobilization adaptors to Fer (aPD-L1-Z-Fer) (FIG. 2A). Although there are various options for NCs that can deliver aPD-L1 with engineered Z, MRI visible Fer was used for potential future translation of this approach to treat HCC. Z adaptor-mediated site-specific immobilization of aPD-L1 on the Fer (Z-Fer) surface exposes the PD-L1 binding Fab region of aPD-L1 outwardly while hiding the Fc region inwardly (FIG. 2A). To compare the IC blockade efficiency of aPD-L1-Z-Fer with conventional chemical conjugation, EDC/NHS chemistry crosslinking primary amine residues of aPD-L1 and carboxyl residues of Fer was performed to form randomly oriented aPD-L1 conjugated Fer (aPD-L1@Fer) (FIG. 2A).

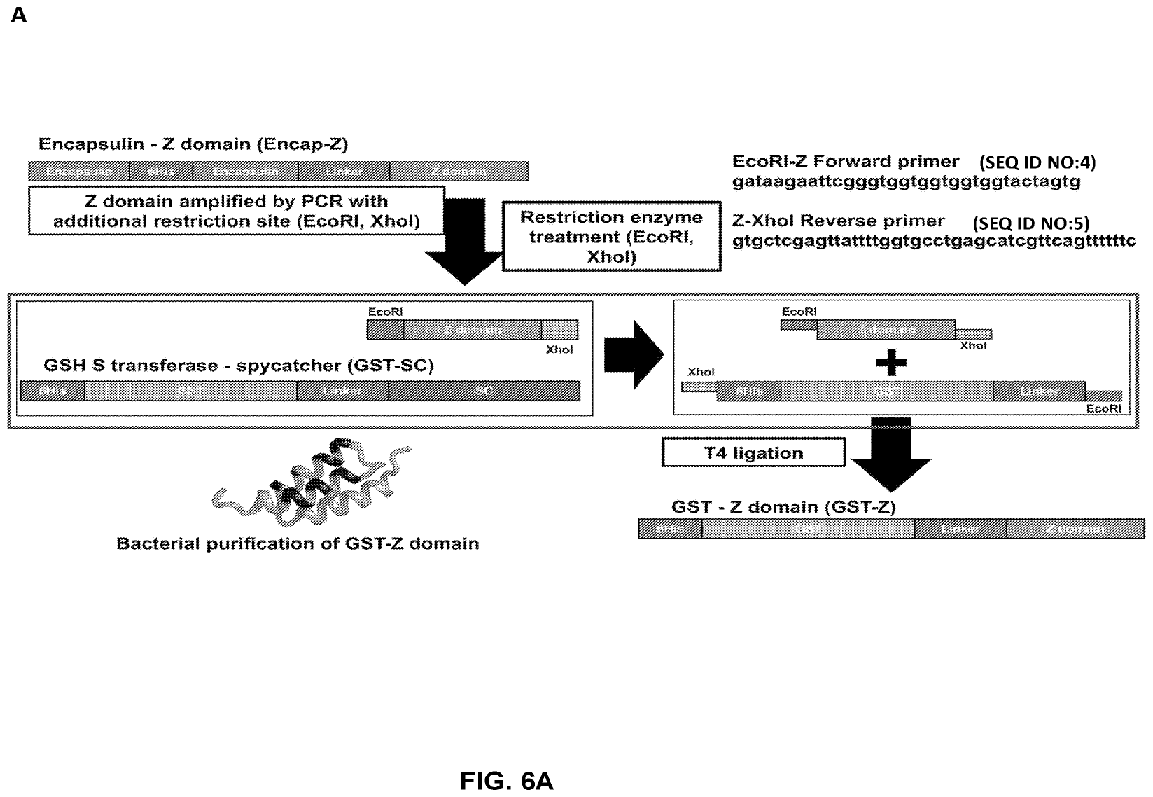

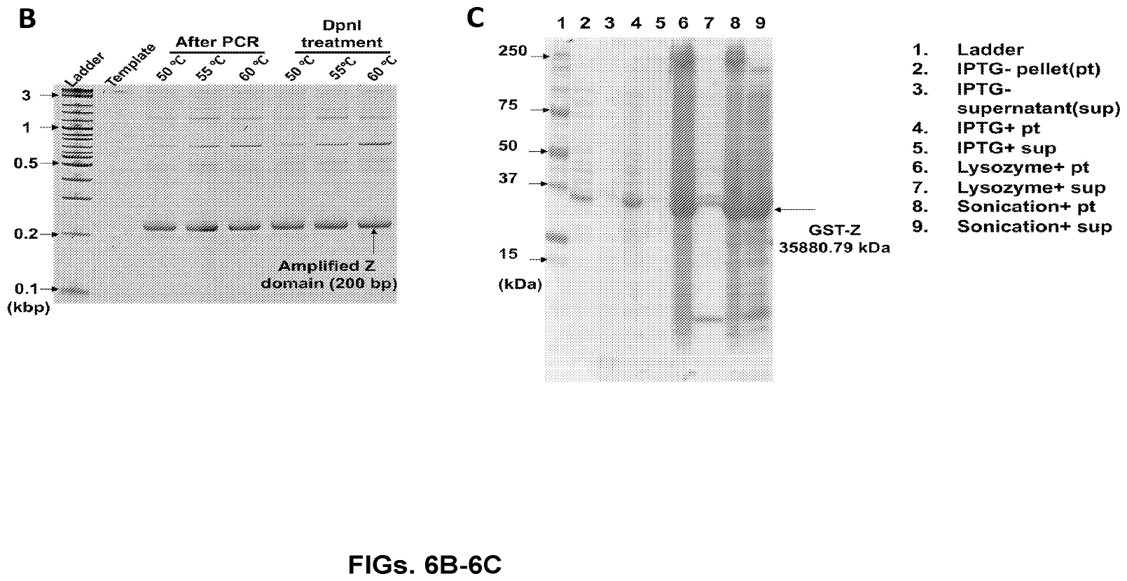

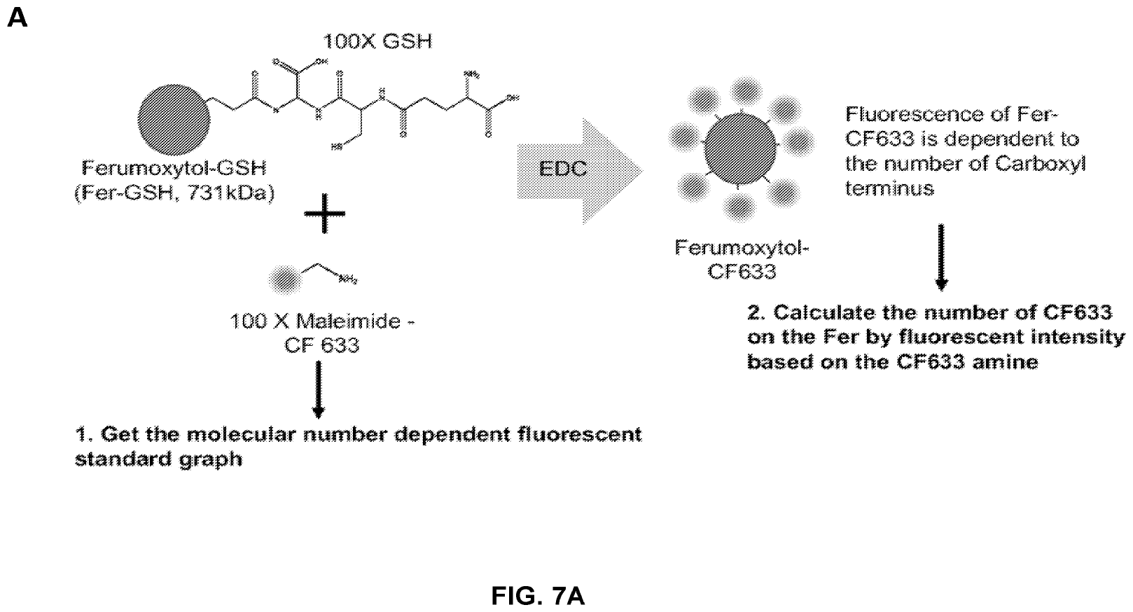

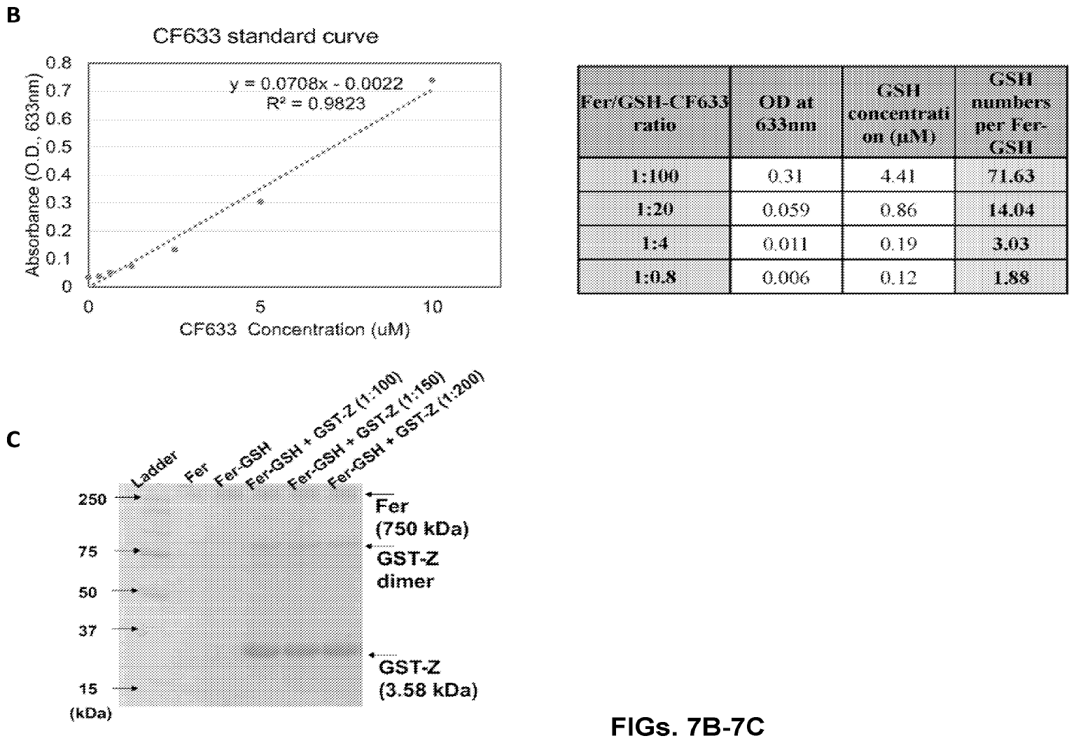

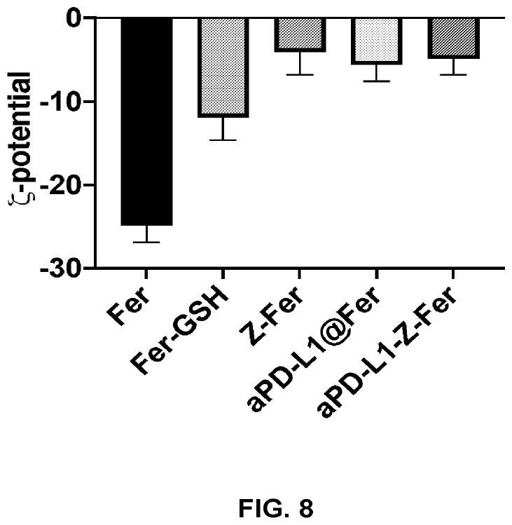

To formulate aPD-L1-Z-Fer, each functionalized Fer and Z was combined with GSH-GST ligand-receptor attachment (Z-Fer) (FIG. 2B). Then a simple co-incubation of aPD-L1 and Z-Fer allowed the preferential connection between Fc region aPD-L1 and Z on Fer (aPD-L1-Z-Fer) (FIG. 2B). For the Z mediated conjugation, GST functionalized Z protein (GST-Z) was engineered as shown in the protein sequence (FIG. 2C, upper). Z sequence was genetically introduced to the posterior position of GST in pET21b plasmid (FIG. 2C (i) and FIG. 6A). Specifically, 200 base pair (bp) sized Z sequence was amplified from Encapsulin-Z (lane 2) with EcoRI bearing forward primer and XhoI backward primer as described in FIGS. 6A and 6B. DpnI enzyme was treated to digest residual template plasmid. Amplified Z was purified by mini-prep column, and purity was confirmed with the 200 bp band in lane 3 in agarose gel electrophoresis (FIG. 2D (i)). GST gene bearing pET21b plasmid and amplified Z was digested with EcoRI and XhoI restriction enzyme (FIG. 2D (ii)). Digested GST template showed decreased band size in lane 4 compared to the intact GST template of lane 2. Then, digested Z inset (lane 3) was mixed and ligated with digested GST template (lane 4) in the presence of the T4 ligase enzyme to formulate the complete plasmid. pET21b plasmid with GST-Z sequence was transformed to the BL21 E. coli for IPTG induced protein overexpression (FIG. 2C (ii)). Solubility of GST-Z in an aqueous solution was evaluated by SDS PAGE of each sample after protein overexpression and bacterial cell lysis. GST-Z band (35.9 kDa) was detected in the supernatant of lane 9 compared to other lanes of supernatant (lanes 3, 5, 7), which represents the GST-Z solubility in aqueous solution (FIG. S1C). Then, the hexahistidine sequence of GST-Z (FIG. 2C, upper) allowed the improved purification with Ni(II) immobilized metal affinity chromatography (FIG. 2C (iii)). Finally, most purified GST-Z was eluted in fractions 1 and 2 (FIG. 2D (iii)). At the same time, the surface of Fer was modified with GSH for ligand-receptor attachment with engineered GST-Z (FIG. 2B). The amount of attached GSH to the surface of Fer was estimated by maleimide-AF633 fluorescence assay (FIG. 7A). The available GSH on Fer was estimated to be 71.6 per particle (FIG. 7B). In SDS PAGE, the attached GST-Z on GSH-Fer was confirmed in the band of lane 4, which size is corresponding to 35.8 kDa (FIG. 7C). 71.6 μmole of GST-Z could be attached to 1 μmole of GSH-Fer to form Z attached Fer (Z-Fer). The hydrodynamic size and surface charge of each Fer, Fer-GSH, and Fer-Z indicated the successful surface modification of Fer (FIG. 7E). The hydrodynamic size was increased from 22 to 36 nm with the attachment of the Z on Fer. The negative surface charge of Fer (−24.9 mV) changed to near neutral (−3.7 mV) by each step of Fer modification (FIG. 8).

Z-Domain Adaptor Mediated aPD-L1 Conjugation with Fer (aPD-L1-Z-Fer)

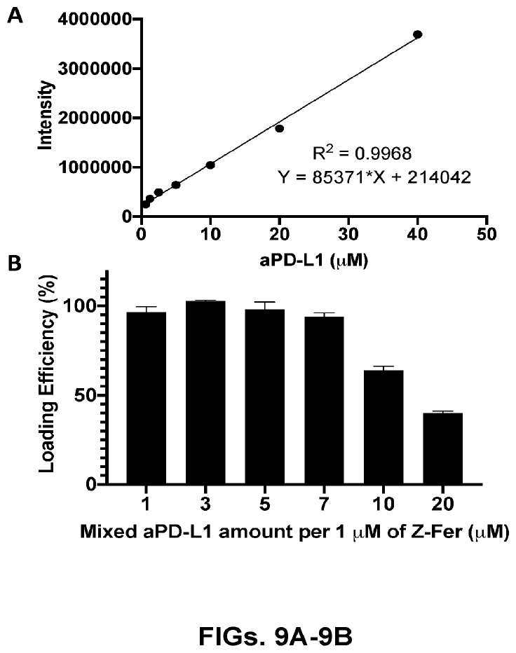

Engineered Z-Fer spontaneously immobilized aPD-L1 ICI mAb into Fer (aPD-L1-Z-Fer) in a simple co-incubation. Fully covered Fer with Z was used for the conjugation of aPD-L1. After the co-incubation with aPD-L1, antibody binding amount was estimated by the intensity of aPD-L1 (lane 7˜13) in nonreducing SDS PAGE (FIGS. 2G and 9A). The measured maximum capacity of Z-Fer for aPD-L1 was limited to 7 units of aPD-L1 (FIGS. 2G and 9B). The maximum capacity might be attributed to the mAb size (10-15 nm)⁶², steric balance⁶³ and the size of Fer. As immobilizing aPD-L1 on Z-Fer, the hydrodynamic size of aPD-L1-Z-Fer (7 units aPD-L1 per Z-Fer) was 62.6 nm which was increased from Z-Fer (32.6 nm) (FIG. 2E). Formulated aPD-L1-Z-Fer showed a strong MRI T2 contrast effect due to Fer. r2 relaxivity of aPD-L1-Z-Fer was 200.6 mM⁻¹s⁻¹ which was similar to Fer (224.2 mM⁻¹s⁻¹) without significant diminish of r2 relaxivity (FIG. 2H).

Enhanced Binding Affinity and Avidity of aPD-L1-Z-Fer in HCC Cells

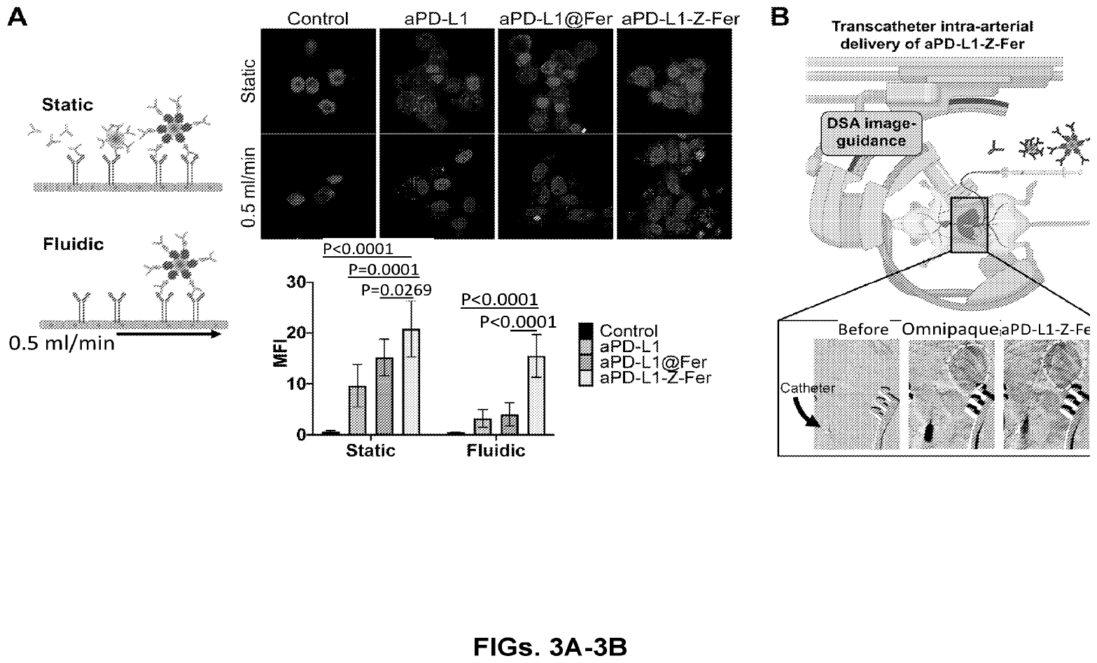

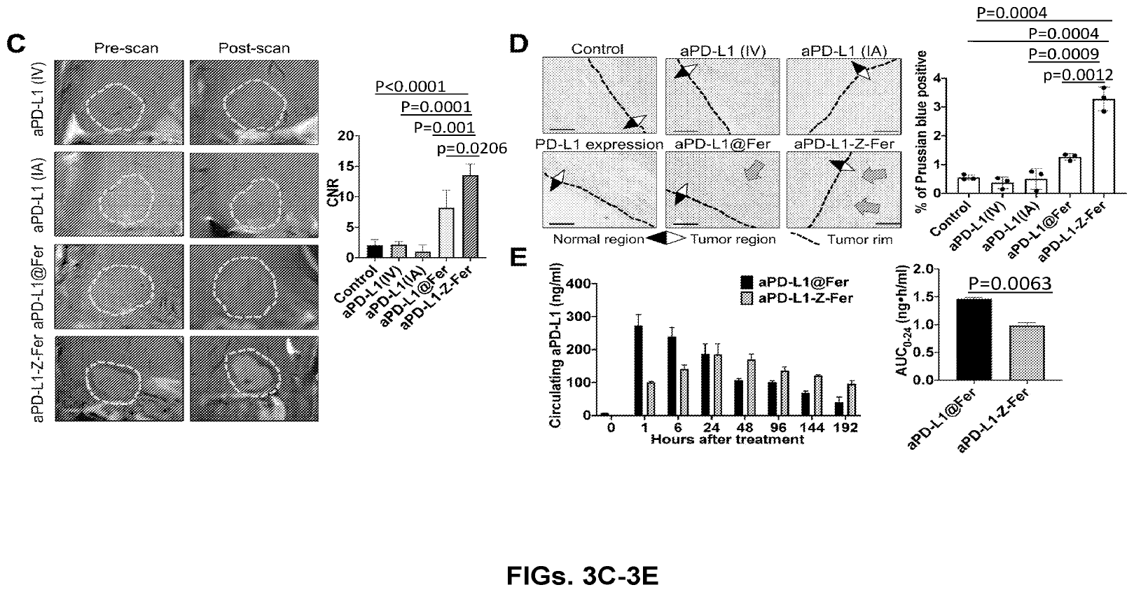

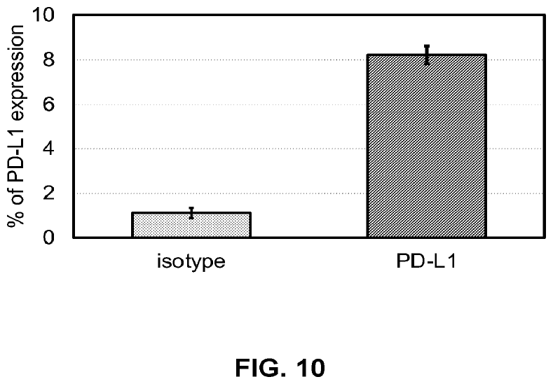

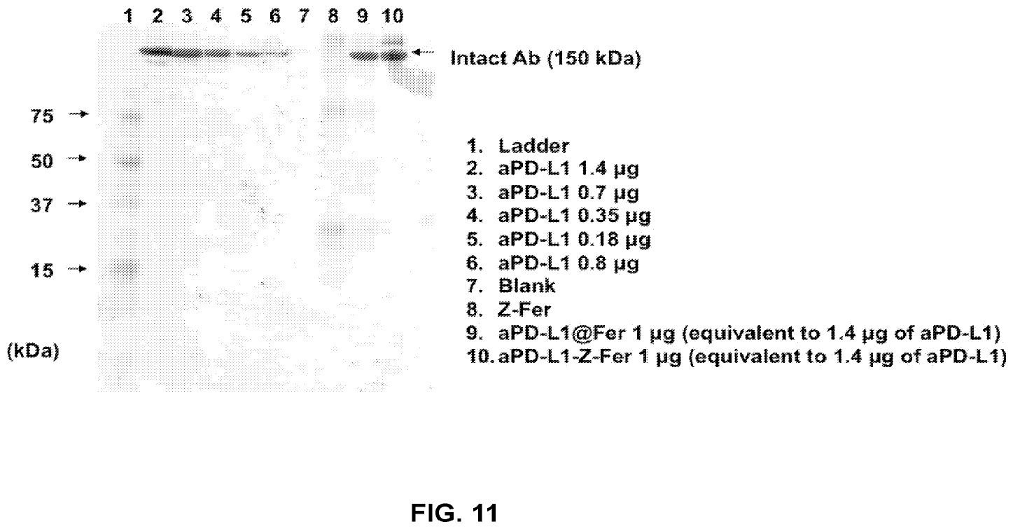

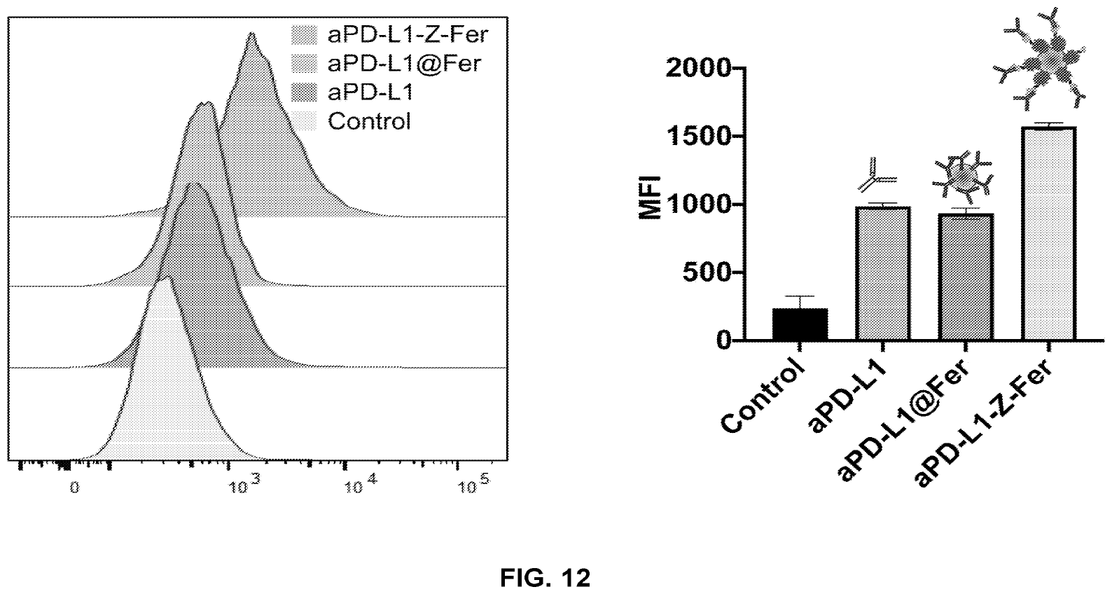

The binding affinity of formulated aPD-L1-Z-Fer was measured in PD-L1 positive N1S1 HCC cells (FIG. 10). To compare the affinity of aPD-L1-Z-Fer with conventional chemical conjugation, well-established EDC/NHS chemistry was used to form chemically conjugated aPD-L1@Fer (FIGS. 2A and 2E). The equivalent 7 units of aPD-L1 were conjugated to one molecule of Fer for each sample (aPD-L1:Fer=7: 1, FIG. 11). Then, the PD-L1 binding affinity of samples was measured using AF488 tagged aPD-L1 (aPD-L1(AF488)) on the samples. To minimize the hindrance of Fc domain to Z interaction and aPD-L1 affinity, AF488 was site-specifically conjugated to N-Glycans of IgG. Next, AF488 tagged samples were co-incubated with PD-L1 positive N1S1 cells. Flowcytometry analysis showed an intense binding signal of aPD-L1-Z-Fer. The mean fluorescence intensity (MFI) of aPD-L1-Z-Fer was amplified approximately 1.5 folds higher than aPD-L1 only or aPD-L1@Fer (FIG. 12). However, aPD-L1@Fer showed a similar MFI with aPD-L1 only (FIG. 12). Z mediated aPD-L1 conjugation on Fer might increase the total affinity of aPD-L1 toward PD-L1 positive N1S1 cells. The enhanced binding affinity of aPD-L1-Z-Fer was further confirmed in a serum flow-cell culture system having a fluidic shear stress of HCC (0.5 ml/min (1.4 dyne/cm²))⁶⁴ and the binding affinity was compared with a static condition. The enhanced binding affinity of aPD-L1-Z-Fer was clearly shown with significantly increased MFI, while the fluorescence intensities of free aPD-L1 and aPD-L1@Fer were intensively compromised by the applied fluidic shear force (FIG. 3A). These results suggest that Z mediated aPD-L1 orientation on Fer (aPD-L1-Z-Fer) has a stronger affinity to hold intact binding between PD-L1 and aPD-L1 than aPD-L1 IgG or aPD-L1@Fer in serum protein solution with fluidics (FIG. 3A). Minimized non-specific protein binding of arranged aPD-L1 on Fer (aPD-L1-Z-Fer) in serum might also enhance the avidity to target PD-L1.³², 65 However, irregular chemical conjugation of aPD-L1 on Fer (aPD-L1@Fer) and undesired domain accessibility of IgG result in non-specific protein binding with exposed Fc region.⁶⁶

In Vivo Transcatheter Hepatic Intra-Arterial Infusion of aPD-L1-Z-Fer

The demonstrated highly efficient binding or blocking affinity of aPD-L1-Z-Fer to PD-L1 could be particularly beneficial for catheter directed local immunotherapy of immune-suppressive HCC. Since HCC receives most blood supply from the hepatic artery, image-guided transcatheter-directed IA delivery allows controlled local delivery of various therapeutics in HCC regions. Here the inventors evaluate that IA infusion of aPD-L1-Z-Fer enhances PD-L1 blockade efficiency and successfully modulates immune-suppressive TME for the treatment of HCC. Firstly, in vivo targeting and binding efficacy of IA infused aPD-L1-Z-Fer was observed with MRI, histology, and pharmacokinetics. aPD-L1-Z-Fer or aPD-L1@Fer (equivalent amount to 1 mg/kg aPD-L1) was delivered to rat N1S1 HCC tumor via digital subtraction angiography (DSA) assisted transcatheter IA infusion (FIG. 3B). As a control, 1 mg/kg concentration of aPD-L1 was systemically injected into the tail vein (IV). Approximately 1 hour after IA infusion of aPD-L1-Z-Fer, MR T2 weight images clearly showed significant signal reduction around the rim of the tumor, indicating accumulated aPD-L1-Z-Fer. However, T2 signal reduction in the groups of IA infusion of aPD-L1-Z-Fer (CNR, 13.6) was much higher than aPD-L1@Fer (CNR, 8.2) (FIG. 3C). Prussian blue histology analysis further confirmed high localization of IA-infused aPD-L1-Z-Fer (FIG. 3D). IA infused aPD-L1-Z-Fer showed increased positive Prussian blue signal (3.28%) around the HCC than aPD-L1@Fer (1.27%) (FIG. 3D). Investigation of circulating aPD-L1 in blood serum after IA infusion of samples demonstrated the retention of locally delivered aPD-L1 using aPD-L1-Z-Fer. The area under the plasma concentration-time curve over the 24 hours (AUCO-24) of non-binding aPD-L1 in the blood was 1.46 mg h/mL in the IA infusion of aPD-L1-Z-Fer. aPD-L1@Fer showed 0.99 mg h/mL, which was almost 1.5 folds higher than aPD-L1-Z-Fer (FIG. 3E). Conclusively, in vitro and in vivo evaluation demonstrated enhanced localized aPD-L1 delivery and binding efficiency of aPD-L1-Z-Fer in HCC.

In Vivo HCC Response of IA Infused aPD-L1-Z-Fer

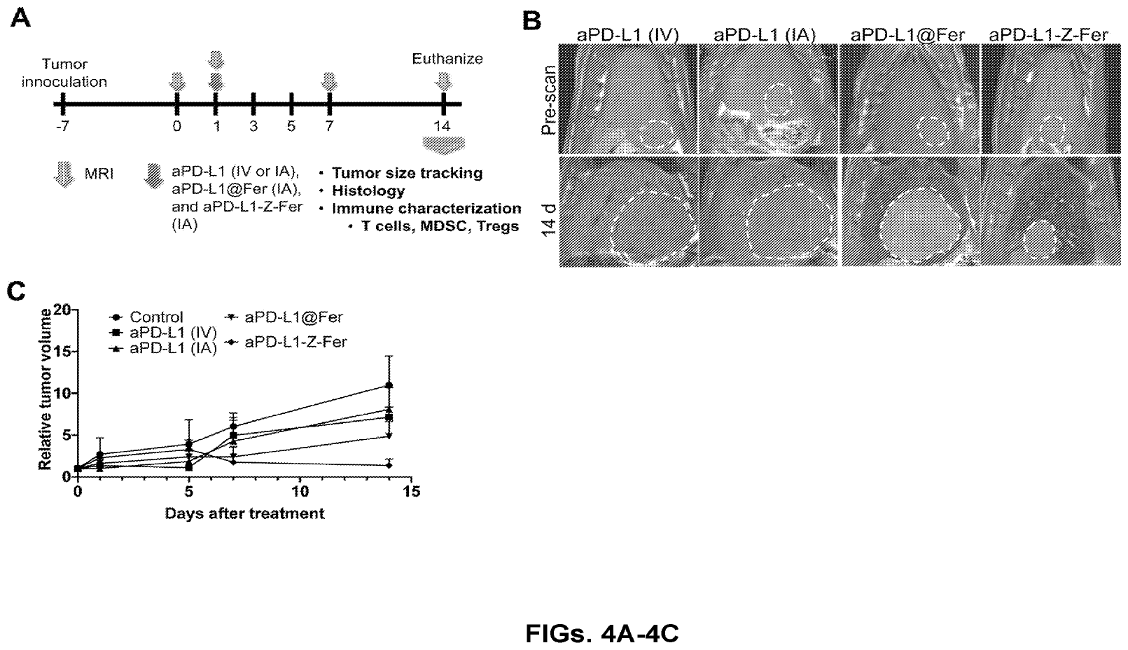

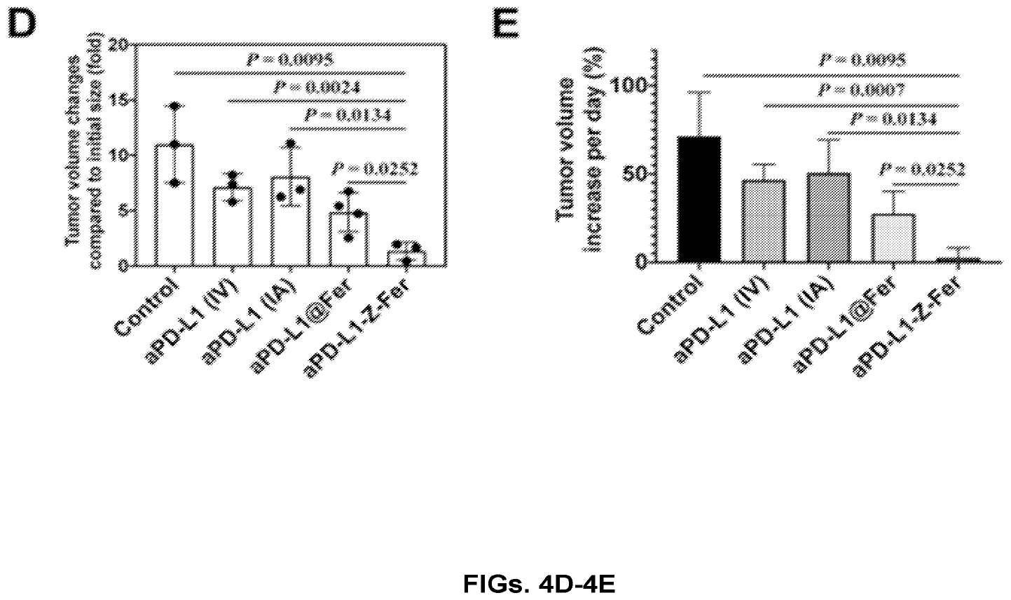

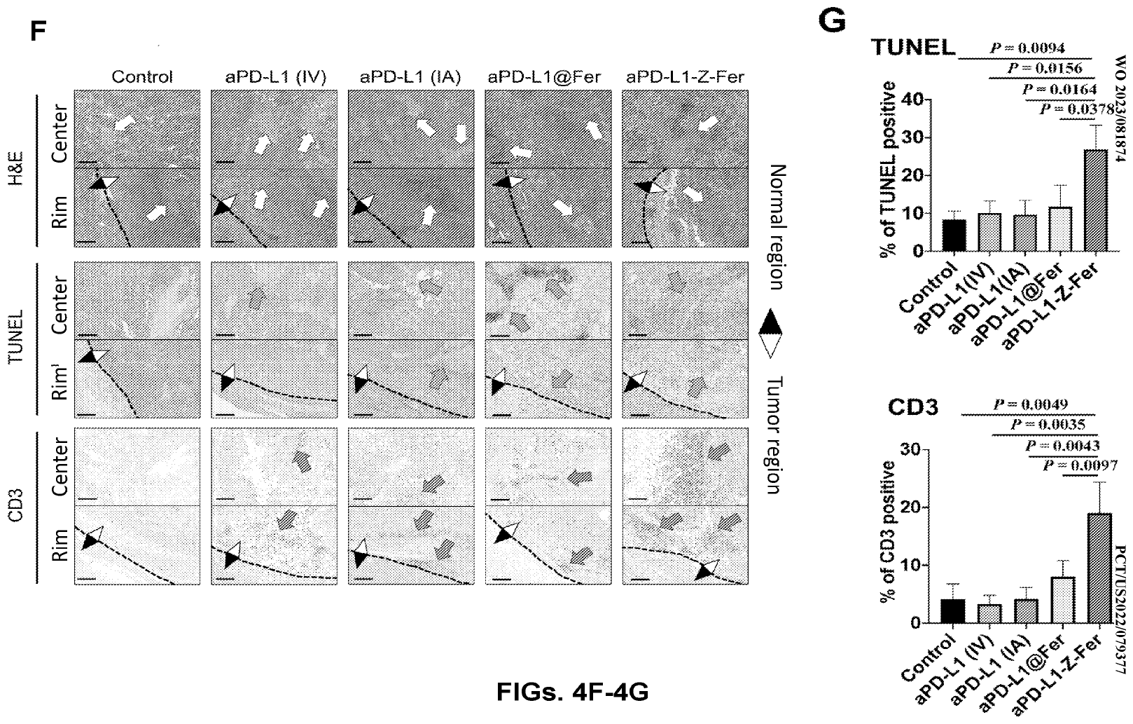

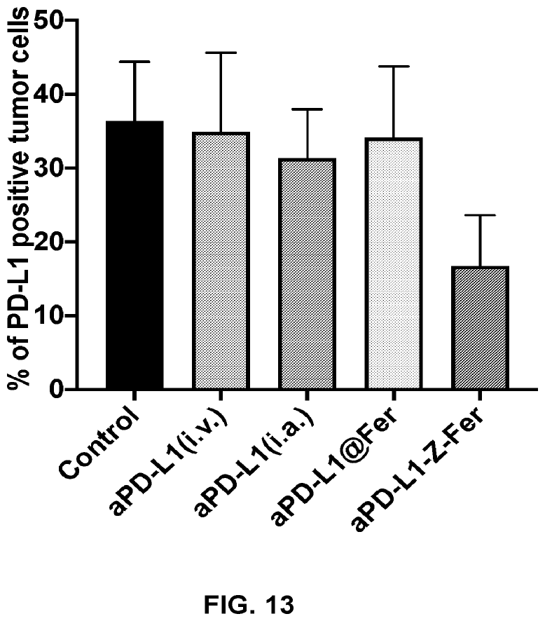

Next, the potential therapeutic efficacy of IA-infused aPD-L1-Z-Fer was evaluated in N1S1 HCC rats. aPD-L1, aPD-L1@Fer, and aPD-L1-Z-Fer (equivalent amount of 10 mg/kg of aPD-L1) were delivered into HCC via hepatic IA injection as described previously (FIG. 4A). Then MRI traced tumor size changes for 14 days. Immune characterization and histological analysis were performed to evaluate the therapeutic efficacy of IA-infused aPD-L1-Z-Fer. As shown in MR images, the tumor growth was significantly suppressed with IA infusion of aPD-L1-Z-Fer compared to aPD-L1 (IV), aPD-L1 (IA), and aPD-L1@Fer (FIGS. 4B and 4C). Relative tumor growth volume changes of the group treated with IA infusion of aPD-L1-Z-Fer compared to other groups were each 8.1, 5.3, 6.0, and 3.6 times less than aPD-L1 (IV), aPD-L1 (IA), and aPD-L1@Fer (FIG. 4D). Also, the tumor growth rate of aPD-L1-Z-Fer was only a 2.6% increase per day, significantly different with aggressive N1S1 tumor growth rate (71.3% per day) in non-treated group (FIG. 4E). H&E and TUNEL stained HCC tissues showed corresponding tumor regression and robust tumor cell death by IA infused aPD-L1-Z-Fer (FIGS. 4F and 4G). Immunohistochemistry of CD3 positive cell staining also demonstrate significant T cell accumulation to the HCC in the local immunotherapy of IA infused aPD-L1-Z-Fer (FIGS. 4F and 4G). Quantitative analysis of TUNEL staining and CD3 positive staining imposes the tumor specific T cell mediated adaptive immune responses (FIGS. 4G and 13). Notably, the same dosage of IV infused aPD-L1 as shown in FIG. 1 or IV infused aPD-L1 was not effective in regressing HCC tumor growth as IA infused aPD-L1-Z-Fer. Local delivery and enhanced binding affinity of aPD-L1-Z-Fer were critical for the anti-cancer therapeutic efficacy of a PD-L1 cancer immunotherapy.

Immune Responses after IA Infusion of aPD-L1-Z-Fer

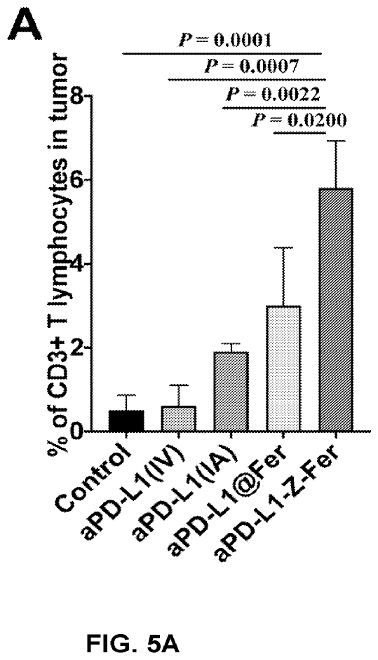

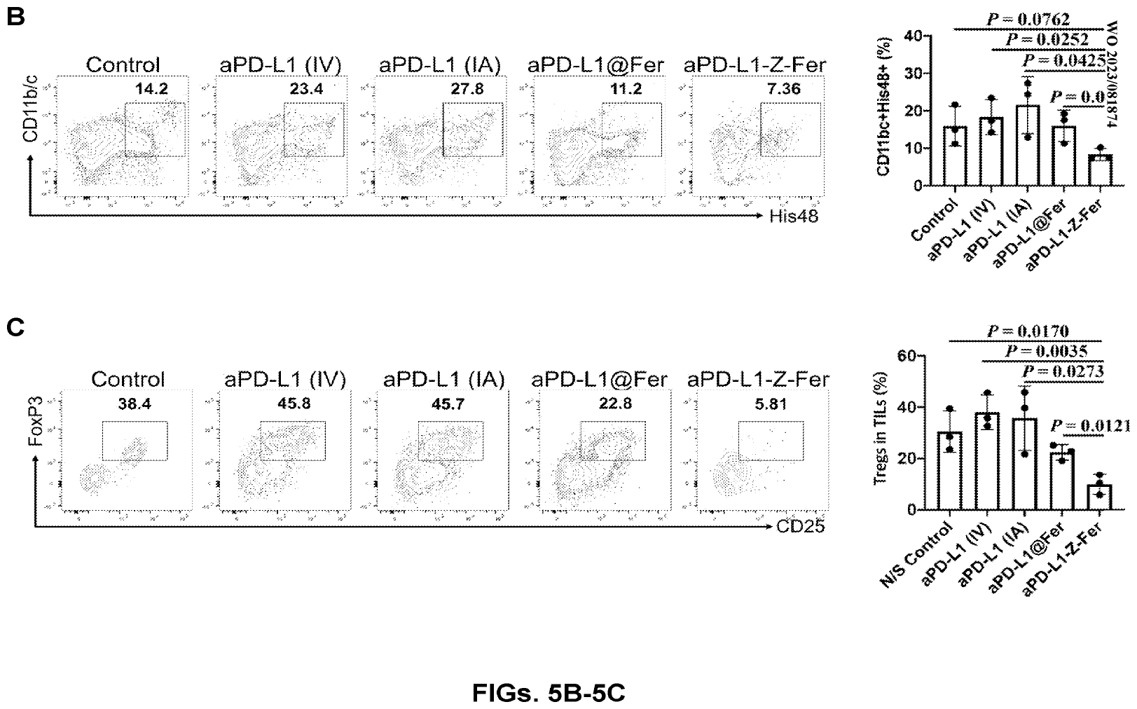

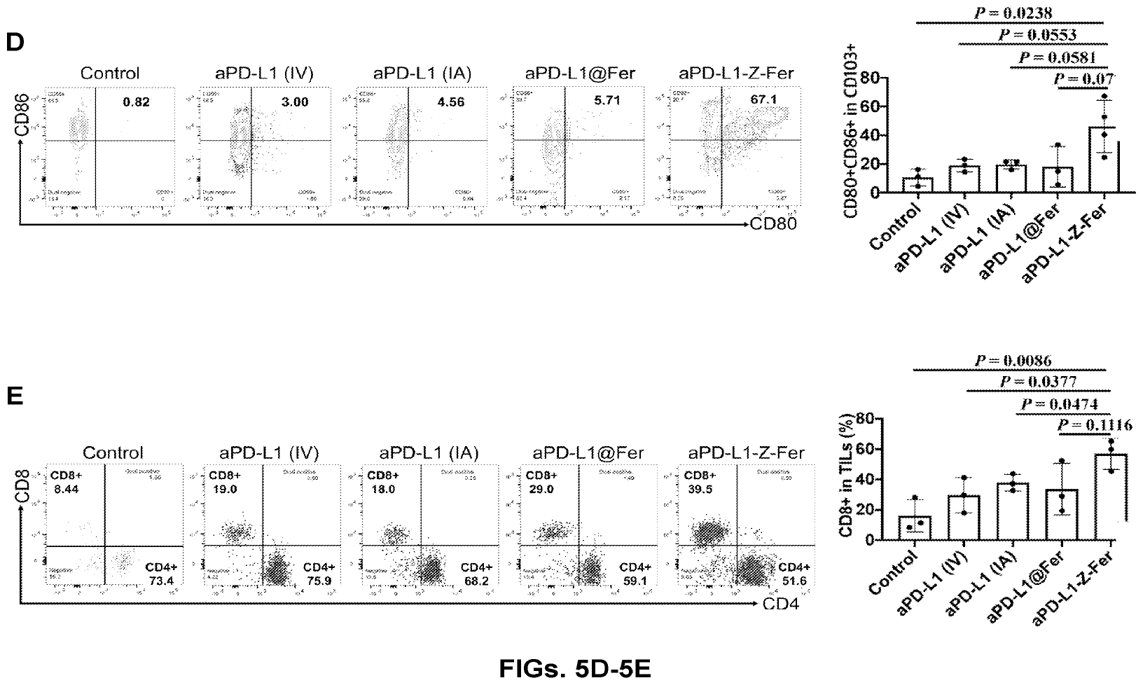

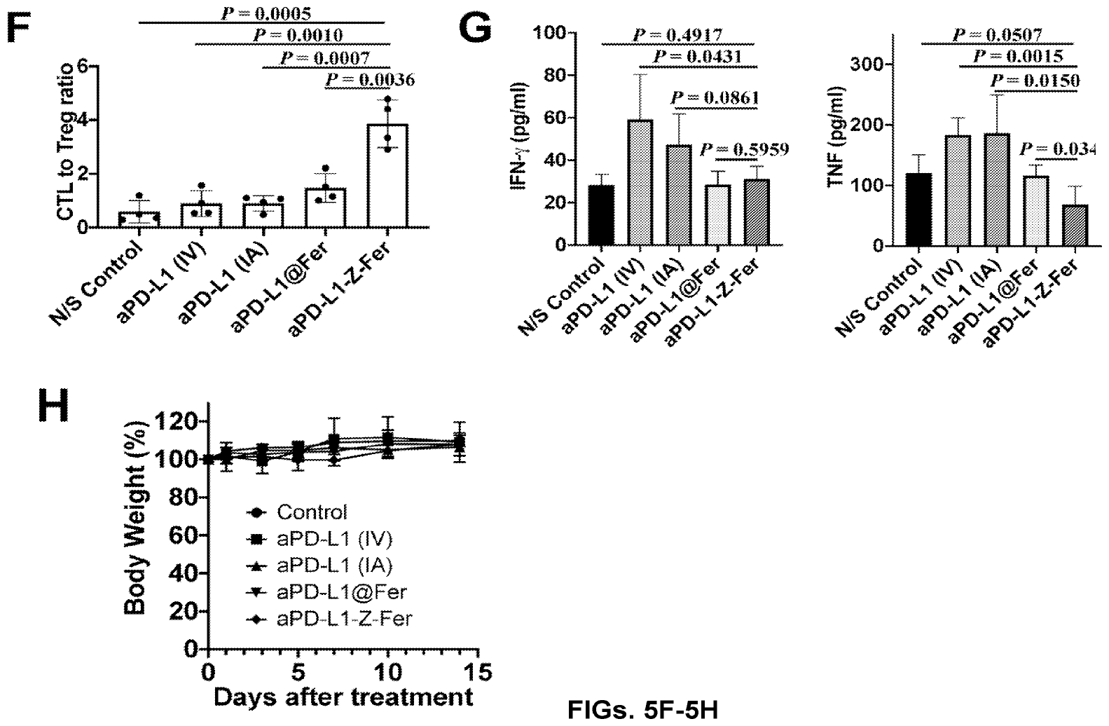

To investigate immune modulation of IA-infused aPD-L1-Z-Fer, main immune-suppressive components in HCC were characterized after each treatment. During the development of immune-escape of HCC, overexpression and activation of PD-1/PD-L1 are key factors for T cell exhaustion by PI3K/AKT pathway activation. Many reports suggest an in-depth association of high PD-1 and PD-L1 expression and poor prognosis of HCC.⁶⁷ These changes recruit various immune suppressor cells to form an immune-suppressive TME. Abundant MDSC by hypoxia in TME is another strong immune suppressor in HCC (FIG. 1D). MDSC also expresses the PD-L1 and produces the IL-10 and TGF-β to promote the Treg infiltration and differentiation. In this regard, effective HCC tumor regression should be accompanied by valid immune conversion after IA infusion of aPD-L1-Z-Fer. Here, an intensive increase of CD3 positive tumor-infiltrating lymphocytes (TILs) to HCC (FIG. 5A) after IA infusion of aPD-L1-Z-Fer (5.8%) was the first clue of the immunogenic conversion of HCC. Then, MDSC population changes were observed from TIL population after 14 days of treatment. As shown in FIG. 5B, the population of MDSC significantly decreased in IA infusion of aPD-L1-Z-Fer treated group compared to that of IA infused free aPD-L1 or aPD-L1@Fer. Moreover, the number of Tregs was intensively compromised after IA infusion of aPD-L1-Z-Fer because aPD-L1 mediated PD-1/PD-L1 axis blocking interferes with the Treg development and infiltration by reduced MDSC population (FIG. 5C). Comparably, IA infused aPD-L1@Fer also showed a slight decrease in MDSC and Treg population, but it was not enough to activate the immune-suppressed TME of HCC. This immunogenic conversion unleashes the maturation of DCs, which are represented by the expression of CD80/CD86 costimulatory molecule expression. As expected, attenuated DC maturation in HCC was outstandingly recovered by locoregional treatments with IA infusion of aPD-L1-Z-Fer (67.2%) compared to other groups (3.0 to 5.7%) (FIG. 5D). Consequently, matured DCs present the TAA presentation to naïve T cells and activate them for HCC specific adaptive immune responses. The anti-cancer immune modulation in HCC induced an increase of functional cytotoxic T lymphocytes (CTLs) in TILs to 21.8% with aPD-L1-Z-Fer treatment while IA infusion of aPD-L1@Fer showed an 8.44% CTLs increase (FIG. 5E). Because the balance of effector CTLs and Tregs is an essential factor in deciding immune responses, the CTL to Treg ratio represents immune activation in HCC. As shown in FIG. 5F, a consequence of the high CTL to Treg ratio after IA infusion of aPD-L1-Z-Fer induced the robust tumor regression.

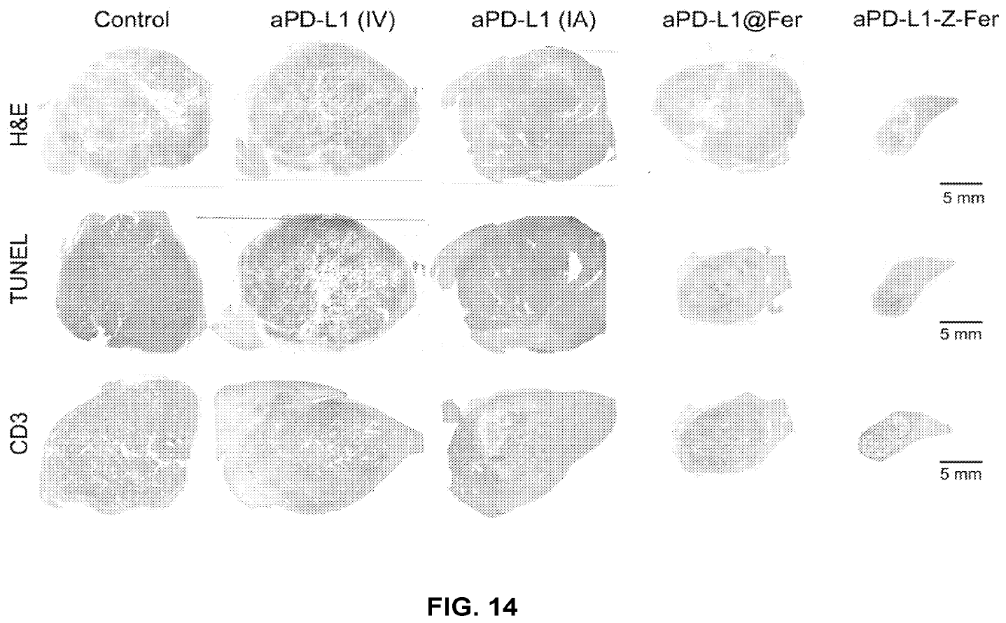

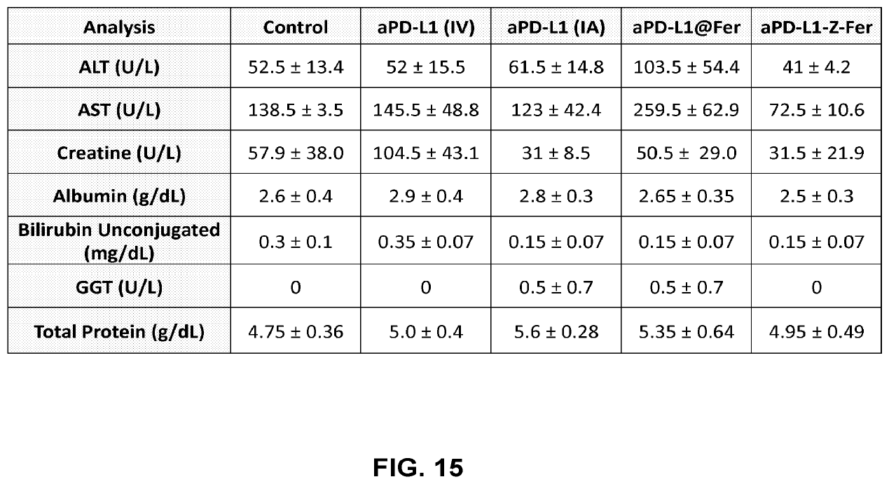

It was noted that the lower affinity of free aPD-L1 and aPD-L1@Fer than that of aPD-L1-Z-Fer might induce the systemic excretion and dose-limiting irAEs. One of the merits of IA-infused aPD-L1-Z-Fer is the reduction of possible systemic circulation with high binding affinity with PD-L1 in HCC. The inventors observed PD-L1 expression level changes within the HCC after each treatment. As shown in FIG. 14, IA infused aPD-L1-Z-Fer enhanced the blockage of aPD-L1-Z-Fer and neutralized the expressed PD-L1 molecules in HCC more efficiently than others. These phenomena also demolish the PD-L1 associated immune-suppressive TME for immunogenic activation corresponding to the tumor regression (FIG. 4C). For irAEs, the increase of TNF in blood serum is regarded as a marker of systemic irAEs along with cytokine storm in the clinic.⁶⁸ Effective binding of IA infused aPD-L1-Z-Fer showed no significant increase of systemic TNF and IFN-γ concentration (FIG. 5G). Bodyweight during 14 days post-treatment was well maintained, indicating no severe systemic irAEs (FIG. 5H). Analysis of the blood serum samples collected at 2 weeks after treatments showed no evidence of a systemic toxicity (FIG. 15). Combined results of systemic cytokine and liver function test suggest the safety of IA infused local aPD-L1-Z-Fer treatment.

### Discussion

Currently, seven types of IgG-based ICI mAbs are licensed as cancer immunotherapy by blocking the PD-1, PD-L1, and CTLA-4 to various solid tumors. Obviously, aPD-L1 cancer immunotherapy potentiates the therapeutic benefits in current clinical trials, but low immunogenic, low mutational, and immune suppressive solid tumors, including HCC, suppress the therapeutic efficacy and avoid the immune surveillance by utilizing immune suppressor cells and TME. Various efforts focusing on combinational aPD-L1 immunotherapies with other standard cancer therapies have been made to improve the efficacy of ICI cancer immunotherapy. However, the therapeutic efficacy is not high as commonly expected. Effective aPD-L1 delivery to the local tumor might be critical to enhance aPD-L1 or aPD-L1 based combination immunotherapies. In this study, HCC was selected as the target disease to be treated with IA-infused aPD-L1-Z-Fer because the development of aPD-L1 immunotherapy is retarded with strong immune suppressive TME. Clinical trial results of aPD-L1 immunotherapy of HCC have not been impressive as much as those of other solid tumors, such as melanoma and lung cancers.⁶⁹ Also, most clinical trials of aPD-L1 immunotherapy are testing the therapeutic efficacy after the first-line Sorafenib chemotherapy. Possible complications and therapeutic resistance with prior standard therapies and damaged immune systems will limit the following systemic dosage of aPD-L1 in the strong immune-suppressive HCC. Enhanced PD-L1 inhibition in local HCC tumors could be a promising approach to achieving higher therapeutic efficacy of aPD-L1 immunotherapy. Here, the inventors proved the importance of oriented ICI mAb with NCs for IC binding affinity and efficacy of ICI mAb. IgG Fc-specific Z adaptor was adopted to conjugate with clinically available Fer for this purpose. Engineered Z adaptor on Fer could site-specifically immobilize the aPD-L1 for outwardly arranged Fab, facilitating the increase of avidity more than randomly conjugated aPD-L1@Fer. Interventional oncology techniques that can approach HCC tumors via blood vessels further allowed clinically feasible local immunotherapy that enhanced the IC blocking efficacy of aPD-L1-Z-Fer in local HCC. Also, the T2 contrast effect of Fer permitted the easy trace of aPD-L1-Z-Fer after the IA infusion. Consequently, IA infused aPD-L1-Z-Fer re-educated the immune-suppressive TME by depleting MDSC and Treg. The effective immune conversion initiated the activation and maturation of DCs against the TAA, resulting in CTL infiltration to HCC and tumoricidal effect. Recently, local immunotherapies, including intra-tumoral or peritumoral immunotherapies, have demonstrated superior anti-cancer immunity with a substantially lower dose used in systemic administration.⁴¹ Clinical trials of tumor-directed immunotherapies that have been largely launched for the translation of local immunotherapies and more advanced approaches combining minimally invasive image-guided injection are expected to be explored. The IA local ICI immunotherapy using aPD-L1-Z-Fer demonstrated herein, effective immune conversion, and anti-cancer immunity provide a new avenue to expand local ICI immunotherapy against HCC. Further, Z attached Fer that can conjugate multiple ICI mAbs and other cancer-targeting mAbs may be used for tumor cell-targeted local immunotherapy and various bi-specific immune modulators.

### Methods

### Cells

N1S1 rat HCC cells (CRL-1604, ATCC, Manassas, VA, U.S.) and McA-RH7777 rat HCC cells (CRL-1601, ATCC, Manassas, VA, U.S.) and were cultured in Iscove's Modified Dulbecco's Medium (IMDM) and Dulbecco's modified Eagle medium (DMEM), respectively. All culturing media were adjusted with 10% fetal bovine serum (Gibco, Grand Island, NY, U.S.) and 100 IU/mL of penicillin/streptavidin (Gibco, Grand Island, NY, U.S.) by ATCC recommendation. Cell culture was maintained in a humidified cell culture incubator (37° C., 5% CO2).

### Animals

Sprague Dawley rats (200 to 300 g body weight) were utilized in this study. (Charles River Laboratories, Wilmington, MA, U.S.) All in vivo experiments in this study were performed in accordance with protocols approved by the Institutional Animal Care and Use Committee (IACUC) of Northwestern University. For the N1S1 HCC model, 3×10⁶ of N1S1 cells in 100 L of PBS was injected to the left lateral lobe of the liver as described before. Briefly, SD rats were anesthetized with 2% isofluorane (Isothesia, Abbot Laboratories, North Chicago, IL, U.S.) with 1 L/min oxygen, preoperative Meloxicam (Loxicom, Norbrook, Newry, Northern Ireland, UK) was then administered at 2 mg/kg subcutaneously. After incision, the liver left lateral lobe was exposed and 3×10⁶ of N1S1 cells in 100 L of PBS was very slowly injected for at least 30 seconds to prevent the backflow. Incision closure was made in two layers using 4-0 absorbable Vetacryl™ sutures (Ethicon, Somerville, NJ, U.S.). After the procedure, Meloxicam (Loxicom, Norbrook, Newry, Northern Ireland, UK) at 2 mg/kg was administered subcutaneously as post-operative analgesia.

### Immune Characterization

After sacrifice, the tumor and spleens were dissected and homogenized by the rubber part of a syringe. Then, samples were sieved to exclude the extra tissue samples using a cell strainer (40 μm, Corning, Corning, NY, U.S.). Erythrocytes were lysed by using ACK lysis buffer (Thermo Fisher, Waltham, MA, U.S.) for 3 to 5 minutes at room temperature. The number of purified cells from tumor and spleens were counted by Countess I (Thermo Fisher, Waltham, MA, U.S.), and 1×10⁶ cells were stained with indicated antibodies (CD3, CD4, CD8, IFN-γ, CD25, FoxP3, CD11b/c, His48, CD103, CD80, and CD86, BD Biosciences, Franklin Lakes, New Jersey, U.S.) to evaluate the immune cell distribution. All flow cytometry analysis was performed using LSR Fortessa II (6 lasers, BD Biosciences, Franklin Lakes, NJ, U.S.).

### Construction and Purification of GST-Z Adapter.

Z-sequence was inserted to the C terminus of GST as described previously.⁷⁰ Z sequence from Encapsulin-Z was amplified with EcoRI-Z forward primer (gataagaattcgggtggtggtggtggtactagtg (SEQ ID NO: 4)) and Z-XhoI reverse primer (gtgctcgagttattttggtgcctgagcatcgttcagttttttc (SEQ ID NO: 5)) by polymerase chain reaction to obtain the Z insert. Prepared Z insert and GST template (pET21b) was digested with EcoRI and XhoI restriction enzyme (New England Biolabs, Ipswich, MA, U.S.) for the reconstruction of GST-Z by T4 ligase. GST-Z in pET21b plasmid was transformed to E. coli (DH5□), New England Biolabs, Ipswich, MA, U.S.) for the amplification and plasmid stabilization. Then, intact GST-Z plasmids were purified by miniprep kit (QIAGEN, Hilden, Germany) to transform the protein expression E. coli (BL21(DE3), New England Biolabs, Ipswich, MA, U.S.) for overexpression of GST-Z by isopropyl β-D-1-thiogalactopyranoside (IPTG, Sigma-Aldrich, St. Louis, MO, U.S.) at RT overnight. Hexahistidine sequence of N-terminus of GST-Z was utilized for the purification by immobilized metal affinity chromatography with Ni-NTA agarose beads (Thermo Fisher, Waltham, MA, U.S.). The concentration and purity of the GST-Z were evaluated with SDS-PAGE and BCA Protein Assay Kit (Thermo Fisher, Waltham, MA, U.S.).

Synthesis and Characterization of Fer-Z, aPD-L1@Fer and aPD-L1-Z-Fer

Carboxyl residues of Fer were conjugated with primary amine residues of GSH by EDC/NHC conjugation chemistry based on the provider's instruction (Thermo Fisher, Waltham, MA, U.S.). GSH decorated Fer (Fer-GSH) was purified by using floating dialysis tubing (MWCO 10K, Spectrum Chemical Mfg. Corp., Gardena, CA, U.S.) to exclude excessive GSH and chemicals. Cysteine of GSH responsive Maleimide-AF633 (Sigma-Aldrich, St. Louis, MO, U.S.) was added to Fer-GSH to quantify the surface GSH amount. Then, 71.68 □mole of purified GST-Z was added to 1 □mole of GSH-Fer to saturate the surface GSH residues for 4 hours, RT. Excessive GST-Z was excluded using GSH coated agarose beads (Thermo Fisher, Waltham, MA, U.S.). The antibody loading efficiency of Fer-Z was evaluated by nonreducing SDS PAGE after purification with floating dialysis tubing for overnight, 4° C. (Molecular weight cut-off (300K), Spectrum Chemical Mfg. Corp., Gardena, CA, U.S.). The intensity of antibody amount was calculated by using Chemi doc (Bio-Rad, Hercules, CA, U.S.) after Coomassie blue staining (FIG. 9). Based on the aPD-L1 standard curve, the attached amount of aPD-L1 was obtained. 6.56 □mole of aPD-L1 (BioXcell, Lebanon, NH, U.S.) could be attached to 1 □mole of Z-Fer as shown in Table. 1. Size changes and C-potential changes of Fer, Fer-GSH, Z-Fer, aPD-L1AFer, and aPD-L1-Z-Fer were measured by DLS analysis (Zetasizer Nano, Malvern Instruments Ltd., Grovewood Road, Malvern, UK).

### Characterization of MRI T2 Contrast.

1% agarose/PBS gel was used as an imaging phantom to characterize the MR imaging contrast effect. Samples were prepared with 0, 10, 20, 30, 40 □g/mL concentration of Fer, aPD-L1@Fer, and aPD-L1-Z-Fer to calculate the R2 relaxivity by using Bruker 7.0T ClinScan high-field small animal MRI (Bruker BioSpin, Ettlingen, Germany).

In Vitro aPD-L1 Affinity Test

AF488 tagged aPD-L1 (aPD-L1(AF488)) was used to evaluate the PD-L1 binding affinity of synthesized aPD-L1@Fer and aPD-L1-Z-Fer. To minimize the hindrance of Fc domain to Z interaction and aPD-L1 affinity, N-Glycans of Fc domains were site-specifically modified with AF488 based on the provider's instruction (Thermo Fisher, Waltham, MA, U.S.). 1×10⁵ cells of N1S1 were incubated with 10 □g of aPD-L1(AF488), aPD-L1(AF488)@Fer, and aPD-L1(AF488)-Z-Fer for 30 min, 4° C. Cells were washed with cold PBS for 3 times to exclude residual aPD-L1 affinity molecules, then analyzed by Flowcytometry (LSR Fortessa II (6 lasers), BD Biosciences, Franklin Lakes, NJ, U.S.).

Additionally, the inventors utilized PD-L1 positive McA-RH7777 adherent rat HCC cell lines to evaluate the enhancement of affinity in physiological condition. 1×10³ cells of McA-RH7777 were pre-incubated to stabilize and attach to the surface of μ-Slide VI 0.5 Glass Bottom (ibidi GmbH, Martinsried, Planegg, Germany) for 1 days. Then, 0.5 mL/min fluidics were applied with a syringe pump to mimic the fluidic shear stress of HCC (1.4 dyne/cm²)⁶⁴ based on the provider's equation.

$$τ = η \cdot 104.7 \cdot Φ$$

$$SShearstress t = \frac{dyn}{{cm}^{2}}$$

$$Dynamic viscosity η = \frac{dyn \times s}{{cm}^{2}}$$

$$Flow rate Φ = \frac{ml}{min}$$

Then, cell culture media with aPD-L1(AF488), aPD-L1(AF488)@Fer, and aPD-L1(AF488)-Z-Fer corresponding to 100 nM of aPD-L1 were exchanged to test the affinity in physiological condition. After 10 minutes, fluidics was changed to normal complete media to wash out the residual. Samples were gently washed with PBS for static or extra 10 minutes with the warm saline flow (0.5 mL/min). Cells were fixed and permeabilized with BD cytofix/cytoperm kit (BD Biosciences, Franklin Lakes, NJ, U.S.) and DAPI were treated for the staining of the nucleus. Images were obtained with AIR spectral confocal laser scanning microscopy (Nikon, Melville, NY, U.S.).

### In Vivo Image-Guided Locoregional Delivery

NiS1 HCC bearing SD rats were used in this experiment. Rats were anesthetized as described in the animal section. After incision, liver lobes were gently flip out from the abdomen to expose the hepatic artery. After gentle dissection from the bile duct and connective tissues, the gastroduodenal artery and common hepatic artery were tied with a silk suture and bulldog clamp. 24½ G angiocatheter (SURFLASH, Terumo Medical Co, Somerset, NJ, U.S.) was gently inserted into the proper hepatic artery. 2 mL of Omnipaque, x-ray contrast agent was slowly injected, and images were obtained by the digital subtraction angiography (DSA, OEC one, GE healthcare, Chicago, IL, U.S.). Then, aPD-L1, aPD-L1@Fer, aPD-L1-Z-Fer corresponding to 1 or 10 mg/kg of aPD-L1 was injected directly to the proper hepatic artery. Incision closure and post-operative support were performed as described in the animal section. After day 5, day 7 and day 14, NiS1 HCC size changes after locoregional delivery was traced by MR T2 scanning with 7 Tesla Bruker Clinscan (Bruker, Billerica, MA, U.S.).

In vivo evaluation of the affinity of aPD-L1@Fer and aPD-L1-Z-Fer

After locoregional delivery of aPD-L1@Fer and aPD-L1-Z-Fer corresponding to 1 mg/kg of aPD-L1, blood serums were harvested from rat tail vein to evaluate circulating aPD-L1 amount each day for 8 days. Amount of aPD-L1 IgG2b (mouse origin) were quantified with IgG ELISA kit (abcam, Cambridge, United Kingdom).

### Quantification of Serum Cytokines.

AA rat cytometric beads array (CBA) inflammation kits (IL-10, IFN-γ, and TNF-α, BD Biosciences, Franklin Lakes, NJ, U.S.) were utilized to quantify cytokines from in vivo serum samples. Blood samples were harvested from the tail vein by 24 G syringe (BD Biosciences, Franklin Lakes, NJ, U.S.) and spun down for serum separation. Blood sera were diluted by five times with assay buffer provided by a rat inflammation kit. Further processes were followed by the manufacturer's protocol and analyzed by flow cytometry and spectrophotometry.

### Histology.

Hematoxylin and eosin (H&E) staining, Prussian blue staining, and terminal deoxynucleotidyl transferase dUTP nick end labeling (TUNEL) staining were performed to evaluate the systemic toxicity of the main organs, including heart, kidney, lung, and liver after 14 days of in vivo experiments. Rats were euthanized, and organs were harvested to fix with 10% neutral formalin solution before histological staining. All histological quantitative analysis were done by using ImageJ (U. S. National Institutes of Health, Bethesda, MD, U.S.) and Qupath software.⁷¹

### Statistical Information.

Analysis results were presented as mean±standard deviation (SD), representing at least three independent experiments. Two-tailed student's t-test and applied to evaluate the significance of experiments. The inventors used GraphPad Prism (La Jolla, CA, U.S.) to analyze the experimental data. Statistical significance was set at *p<0.05; **p<0.01; ***p<0.001, as indicated in the figure legends.

### REFERENCES

1. Kew M C. Epidemiology of hepatocellular carcinoma. Toxicology 181-182, 35-38 (2002).
2. Kim E, Viatour P. Hepatocellular carcinoma: old friends and new tricks. Exp Mol Med 52, 1898-1907 (2020).
3. Chow P K, et al. High-dose tamoxifen in the treatment of inoperable hepatocellular carcinoma: A multicenter randomized controlled trial. Hepatology 36, 1221-1226 (2002).
4. Lai C L, Wu P C, Chan G C, Lok A S, Lin H J. Doxorubicin versus no antitumor therapy in inoperable hepatocellular carcinoma. A prospective randomized trial. Cancer 62, 479-483 (1988).
5. Ahmed M, Goldberg S N. Thermal ablation therapy for hepatocellular carcinoma. Journal of Vascular & Interventional Radiology 13, S231-244 (2002).
6. Clark T W, Soulen M C. Chemical ablation of hepatocellular carcinoma. J Vasc Interv Radiol 13, S245-252 (2002).
7. Ackerman N B. Experimental studies on the circulation dynamics of intrahepatic tumor blood supply. Cancer 29, 435-439 (1972).
8. Marelli L, et al. Transarterial therapy for hepatocellular carcinoma: which technique is more effective? A systematic review of cohort and randomized studies. Cardiovasc Intervent Radiol 30, 6-25 (2007).
9. Biolato M, et al. Transarterial chemoembolization (TACE) for unresectable HCC: a new life begins? Eur Rev Med Pharmacol Sci 14, 356-362 (2010).
10. Yarchoan M, et al. PD-L1 expression and tumor mutational burden are independent biomarkers in most cancers. Jci Insight 4, (2019).
11. Kudo M. Immune Checkpoint Blockade in Hepatocellular Carcinoma. Liver Cancer 4, 201-207 (2015).
12. El-Khoueiry A B, et al. Phase I/II safety and antitumor activity of nivolumab in patients with advanced hepatocellular carcinoma (HCC): CA209-040. Journal of Clinical Oncology 33, (2015).
13. Rizvi N A, et al. Activity and safety of nivolumab, an anti-PD-1 immune checkpoint inhibitor, for patients with advanced, refractory squamous non-small-cell lung cancer (CheckMate 063): a phase 2, single-arm trial. Lancet Oncol 16, 257-265 (2015).
14. Prieto J, Melero I, Sangro B. Immunological landscape and immunotherapy of hepatocellular carcinoma. Nat Rev Gastroenterol Hepatol 12, 681-700 (2015).
15. Greten T F, Wang X W, Korangy F. Current concepts of immune based treatments for patients with HCC: from basic science to novel treatment approaches. Gut 64, 842-848 (2015).
16. Tsuchiya N, Sawada Y, Endo I, Uemura Y, Nakatsura T. Potentiality of immunotherapy against hepatocellular carcinoma. World J Gastroenterol 21, 10314-10326 (2015).
17. Centanni M, Moes D, Troconiz I F, Ciccolini J, van Hasselt J G C. Clinical Pharmacokinetics and Pharmacodynamics of Immune Checkpoint Inhibitors. Clin Pharmacokinet 58, 835-857 (2019).
18. Keizer R J, Huitema A D, Schellens J H, Beijnen J H. Clinical pharmacokinetics of therapeutic monoclonal antibodies. Clin Pharmacokinet 49, 493-507 (2010).
19. Riley R S, June C H, Langer R, Mitchell M J. Delivery technologies for cancer immunotherapy.
20. Nat Rev Drug Discov 18, 175-196 (2019).
21. Mitchell M J, Billingsley M M, Haley R M, Wechsler M E, Peppas N A, Langer R. Engineering precision nanoparticles for drug delivery. Nature Reviews Drug Discovery 20, 101-124 (2021).
22. Irvine D J, Dane E L. Enhancing cancer immunotherapy with nanomedicine. Nature Reviews Immunology 20, 321-334 (2020).
23. Martin J D, Cabral H, Stylianopoulos T, Jain R K. Improving cancer immunotherapy using nanomedicines: progress, opportunities and challenges. Nature Reviews Clinical Oncology 17, 251-266 (2020).
24. Wang D, et al. Engineering nanoparticles to locally activate T cells in the tumor microenvironment. Sci Immunol 4, (2019).
25. Bu J Y, et al. An Avidity-Based PD-L1 Antagonist Using Nanoparticle-Antibody Conjugates for Enhanced Immunotherapy. Nano Lett 20, 4901-4909 (2020).
26. Jiang C T, et al. Immunomodulating nano-adaptors potentiate antibody-based cancer immunotherapy. Nat Commun 12, (2021).
27. Wilhelm S, et al. Analysis of nanoparticle delivery to tumours. Nat Rev Mater 1, (2016).
28. Blanco E, Shen H, Ferrari M. Principles of nanoparticle design for overcoming biological barriers to drug delivery. Nature Biotechnology 33, 941-951 (2015).
29. Bertrand N, et al. Mechanistic understanding of in vivo protein corona formation on polymeric nanoparticles and impact on pharmacokinetics. Nat Commun 8, (2017).
30. Desnoyer A, et al. Pharmacokinetic/pharmacodynamic relationship of therapeutic monoclonal antibodies used in oncology: Part 2, immune checkpoint inhibitor antibodies. Eur J Cancer 128, 119-128 (2020).
31. Yong K W, Yuen D, Chen M Z, Porter C J H, Johnston A P R. Pointing in the Right Direction: Controlling the Orientation of Proteins on Nanoparticles Improves Targeting Efficiency. Nano Lett 19, 1827-1831 (2019).
32. Kang H J, Kang Y J, Lee Y M, Shin H H, Chung S J, Kang S. Developing an antibody-binding protein cage as a molecular recognition drug modular nanoplatform. Biomaterials 33, 5423-5430 (2012).
33. Tonigold M, et al. Pre-adsorption of antibodies enables targeting of nanocarriers despite a biomolecular corona. Nat Nanotechnol 13, 862-869 (2018).
34. Iijima M, Araki K, Liu Q, Somiya M, Kuroda S. Oriented immobilization to nanoparticles enhanced the therapeutic efficacy of antibody drugs. Acta Biomater 86, 373-380 (2019).
35. Shim G, Kim D, Lee S, Chang R S, Byun J, Oh Y K. Staphylococcus aureus-mimetic control of antibody orientation on nanoparticles. Nanomedicine 16, 267-277 (2019).
36. Bonroy K, et al. Comparison of random and oriented immobilisation of antibody fragments on mixed self-assembled monolayers. J Immunol Methods 312, 167-181 (2006).
37. Blumberg R S, Lencer W I. Antibodies in the breakdown lane. Nature Biotechnology 23, 1232-1234 (2005).
38. Choi B, Choi H, Yu B, Kim D H. Synergistic Local Combination of Radiation and Anti-Programmed Death Ligand 1 Immunotherapy Using Radiation-Responsive Splintery Metallic Nanocarriers. ACS Nano, (2020).
39. Choi B, Jung H, Yu B, Choi H, Lee J, Kim D H. Sequential M R Image-Guided Local Immune Checkpoint Blockade Cancer Immunotherapy Using Ferumoxytol Capped Ultralarge Pore Mesoporous Silica Carriers after Standard Chemotherapy. Small 15, e1904378 (2019).
40. Yu B, Choi B S, Li W G, Kim D H. Magnetic field boosted ferroptosis-like cell death and responsive MRI using hybrid vesicles for cancer immunotherapy. Nat Commun 11, (2020).
41. Yu B, Zhang W T, Kwak K J, Choi H J, Kim D H. Electric Pulse Responsive Magnetic Nanoclusters Loaded with Indoleamine 2,3-Dioxygenase Inhibitor for Synergistic Immuno-Ablation Cancer Therapy. Acs Appl Mater Inter 12, 54415-54425 (2020).
42. Murthy V, Minehart J, Sterman D H. Local Immunotherapy of Cancer: Innovative Approaches to Harnessing Tumor-Specific Immune Responses. J Natl Cancer Inst 109, (2017).
43. Choi B, Choi H, Yu B, Kim D H. Synergistic Local Combination of Radiation and Anti-Programmed Death Ligand 1 Immunotherapy Using Radiation-Responsive Splintery Metallic Nanocarriers. ACS Nano 14, 13115-13126 (2020).
44. Choi B, Jung H, Yu B, Choi H, Lee J, Kim D H. Sequential M R Image-Guided Local Immune Checkpoint Blockade Cancer Immunotherapy Using Ferumoxytol Capped Ultralarge Pore Mesoporous Silica Carriers after Standard Chemotherapy. Small 15, (2019).
45. Kim D H, et al. Multimodal Imaging of Nanocomposite Microspheres for Transcatheter Intra-Arterial Drug Delivery to Liver Tumors. Sci Rep 6, 29653 (2016).
46. Jeon M J, Gordon A C, Larson A C, Chung J W, Kim Y I, Kim D H. Transcatheter intra-arterial infusion of doxorubicin loaded porous magnetic nano-clusters with iodinated oil for the treatment of liver cancer. Biomaterials 88, 25-33 (2016).
47. Kim D H, Chen J, Omary R A, Larson A C. MRI visible drug eluting magnetic microspheres for transcatheter intra-arterial delivery to liver tumors. Theranostics 5, 477-488 (2015).
48. Chen J, et al. Poly(lactide-co-glycolide) microspheres for MRI-monitored delivery of sorafenib in a rabbit VX2 model. Biomaterials 61, 299-306 (2015).
49. Prieto J, Melero I, Sangro B. Immunological landscape and immunotherapy of hepatocellular carcinoma (vol 12, pg 681, 2015). Nat Rev Gastro Hepat 12, (2015).
50. Desrichard A, Snyder A, Chan T A. Cancer Neoantigens and Applications for Immunotherapy.
51. Clin Cancer Res 22, 807-812 (2016).
52. Schumacher T N, Schreiber R D. Neoantigens in cancer immunotherapy. Science 348, 69-74 (2015).
53. Iyer R V, et al. Dose-Dependent Sorafenib-Induced Immunosuppression Is Associated with Aberrant NFAT Activation and Expression of PD-1 in T Cells. Cancers (Basel) 11, (2019).
54. Voron T, et al. VEGF-A modulates expression of inhibitory checkpoints on CD8+ T cells in tumors. J Exp Med 212, 139-148 (2015).
55. Schoenfeld A J, Hellmann M D. Acquired Resistance to Immune Checkpoint Inhibitors. Cancer Cell 37, 443-455 (2020).
56. Huang M, et al. The Influence of Immune Heterogeneity on the Effectiveness of Immune Checkpoint Inhibitors in Multifocal Hepatocellular Carcinomas. Clin Cancer Res 26, 4947-4957 (2020).
57. Zemek R M, Chin W L, Nowak A K, Millward M J, Lake R A, Lesterhuis W J. Sensitizing the Tumor Microenvironment to Immune Checkpoint Therapy. Front Immunol 11, 223 (2020).
58. Pol J, et al. Trial Watch: Immunogenic cell death inducers for anticancer chemotherapy.
59. Oncoimmunology 4, e1008866 (2015).
60. Brahmer J R, et al. Safety and activity of anti-PD-L1 antibody in patients with advanced cancer. N Engl J Med 366, 2455-2465 (2012).
61. Sangro B, Sarobe P, Hervas-Stubbs S, Melero I. Advances in immunotherapy for hepatocellular carcinoma. Nat Rev Gastroenterol Hepatol, (2021).
62. Iwata T, et al. PD-L1(+) MDSCs are increased in HCC patients and induced by soluble factor in the tumor microenvironment. Sci Rep-Uk 6, (2016).
63. Wu K, Kryczek I, Chen L P, Zou W P, Welling T H. Kupffer Cell Suppression of CD8(+) T Cells in Human Hepatocellular Carcinoma Is Mediated by B7-H1/Programmed Death-1 Interactions. Cancer Res 69, 8067-8075 (2009).
64. Picardo S L, Doi J, Hansen A R. Structure and Optimization of Checkpoint Inhibitors. Cancers (Basel) 12, (2019).
65. Vidarsson G, Dekkers G, Rispens T. IgG subclasses and allotypes: from structure to effector functions. Front Immunol 5, 520 (2014).
66. Saha B, Songe P, Evers T H, Prins M W J. The influence of covalent immobilization conditions on antibody accessibility on nanoparticles. Analyst 142, 4247-4256 (2017).
67. Wang X, et al. Fluid Shear Stress Promotes Autophagy in Hepatocellular Carcinoma Cells. Int J Biol Sci 14, 1277-1290 (2018).
68. Ruiz G, Tripathi K, Okyem S, Driskell J D. pH Impacts the Orientation of Antibody Adsorbed onto Gold Nanoparticles. Bioconjug Chem 30, 1182-1191 (2019).
69. Driskell J, Ruiz G, Tripathi K. Impact of pH on the orientation of antibody adsorbed onto gold nanoparticles. Abstr Pap Am Chem S 257, (2019).
70. Pinato D J, et al. Immune-based therapies for hepatocellular carcinoma. Oncogene 39, 3620-3637 (2020).
71. Chen X, Song X, Li K, Zhang T. FcgammaR-Binding Is an Important Functional Attribute for Immune Checkpoint Antibodies in Cancer Immunotherapy. Front Immunol 10, 292 (2019).
72. Keenan B P, Fong L, Kelley R K. Immunotherapy in hepatocellular carcinoma: the complex interface between inflammation, fibrosis, and the immune response. Journal for Immunotherapy of Cancer 7, (2019).
73. Choi B, et al. Effective Delivery of Antigen-Encapsulin Nanoparticle Fusions to Dendritic Cells Leads to Antigen-Specific Cytotoxic T Cell Activation and Tumor Rejection. Acs Nano 10, 7339-7350 (2016).
74. Bankhead P, et al. QuPath: Open source software for digital pathology image analysis. Sci Rep 7, 16878 (2017).

1. An iron nanoparticle comprising an antibody directionally attached by the constant region (Fc) of the antibody to the nanoparticle with a directional linker, wherein the antigen binding sites of the antibody are facing outwardly from the nanoparticle.

2. The nanoparticle of claim 1, wherein the nanoparticle comprises ferumoxytol.

3. The nanoparticle of claim 1 or claim 2, wherein an outer surface of the nanoparticle is functionalized with free exposed carboxyl residues chemically linked to glutathione.

4. The nanoparticle of any of the preceding claims, wherein the linker comprises glutathione S transferase (GST) linked to the Z domain of staphylococcal protein A.

5. The nanoparticle of any of the preceding claims, wherein the antibody is an immune checkpoint inhibitor (ICI) antibody.

6. The nanoparticle of any of the preceding claims, wherein the ICI antibody is an anti-PD-L1 antibody.

7. The nanoparticle of any of claim 1-5, wherein the ICI antibody is an anti-PD-1 antibody.

8. The nanoparticle of any of claims 1-7, wherein the nanoparticle has greater binding affinity for the antigen than the antibody alone or a second iron nanoparticle having the antibody chemically linked directly to the second nanoparticle.

9. The nanoparticle of any of claims 1-7, wherein the nanoparticle has increased resistance to fluidic shear force compared to the antibody alone or a second iron nanoparticle having the antibody chemically linked directly to the second nanoparticle.

10. The nanoparticle of any of claims 1-7, wherein the nanoparticle induces increased CD3⁺ cell accumulation in a tumor microenvironment (TME) compared to the antibody alone or a second iron nanoparticle having the antibody chemically linked directly to the second nanoparticle.

11. The nanoparticle of any of claims 1-7, wherein the nanoparticle reduces myeloid derived suppressor cell (MDSC) accumulation in a TME compared to the antibody alone or a second iron nanoparticle having the antibody chemically linked directly to the second nanoparticle.

12. The nanoparticle of any of claims 1-7, wherein the nanoparticle increases dendritic cell (DC) maturation compared to the antibody alone or a second iron nanoparticle having the antibody chemically linked directly to the second nanoparticle.

13. A pharmaceutical composition comprising the nanoparticle of any of the preceding claims and a pharmaceutically acceptable delivery vehicle.

14. A method of treating a subject in need of treatment for cancer, the method comprising administering a therapeutically effective amount of the pharmaceutical composition of claim 13.

15. The method of claim 14, wherein the cancer is hepatocellular carcinoma (HCC)

16. The method of claim 14 or 15, wherein the administering is locoregionally.

17. The method of claim 16, wherein the administering is transarterially.

18. The method of claim 16, wherein the administering is intratumorally.

19. The method of claim 16, wherein the administering is intrahepatically.

20. A method comprising:
conjugating glutathione to an iron nanoparticle;
co-incubating the nanoparticle with a glutathione S-transferase functionalized with a Z domain of staphylococcal protein A; and
co-incubating the nanoparticle with an antibody.

21. The method of claim 20, wherein the iron nanoparticle comprises ferumoxytol.

22. The method of claim 20 or claim 21, wherein prior to the conjugating step, the nanoparticle is functionalized with free exposed carboxyl residues.

23. The method of any of claims 20-22, wherein the antibody is an immune checkpoint inhibitor (ICI).

24. The method of claim 23, wherein the ICI antibody is an anti-PD-L1 antibody.

25. The method of claim 23, wherein the ICI antibody is an anti-PD-1 antibody.


SEQUENCE LISTING (5 sequences)

SEQ ID NO: 1 - protein, 306 aa, synthetic construct
  REGION 1..306: note = Synthetic - GSH S transferase - Z domain (GST-Z)
MGSSHHHHHHSQDPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDKNSGGGTGGGSGGGVDNKFNKEQQNAFYEILHLPNLNEEQRNAFIQSLKDDPSQSANLLAEAKKLNDAQAPK

SEQ ID NO: 2 - protein, 223 aa, synthetic construct
  REGION 1..223: note = Synthetic- Glutathione S transferase (GST)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDKNS

SEQ ID NO: 3 - protein, 58 aa, synthetic construct [sequence omitted]

SEQ ID NO: 4 - DNA, 34 bp, synthetic construct
  misc_feature 1..34: note = Synthetic- EcoRI-Z forward primer
  source 1..34: mol_type = other DNA
gataagaattcgggtggtggtggtggtactagtg

SEQ ID NO: 5 - DNA, 43 bp, synthetic construct
  misc_feature 1..43: note = Synthetic- Z- XhoI reverse primer
  source 1..43: mol_type = other DNA
gtgctcgagttattttggtgcctgagcatcgttcagttttttc